(19) United States
(12) Patent Application Publication
(10) Pub. No.: US 2025/0391136 A1
(43) Pub. Date: Dec. 25, 2025

(54) CONCURRENT ADJUSTING OF TOOTH MODELS

(71) Applicant: EXOCAD GMBH, Darmstadt (DE)

(72) Inventor: Iurie Chiosa, Weiterstadt (DE)

(21) Appl. No.: 18/753,716

(22) Filed: Jun. 25, 2024

(51) Int. Cl. G06T 19/20; A61C 13/00; A61C 13/34; G06F 30/17

(52) U.S. Cl. CPC G06T 19/20; A61C 13/0004; A61C 13/34; G06F 30/17; G06T 2210/41; G06T 2219/2016; G06T 2219/2021

(57) ABSTRACT

Disclosed is a computer-implemented method for adjusting two or more three-dimensional digital tooth models of a tooth set of three-dimensional digital tooth models of a jaw. The tooth set is a tooth set for a dental restoration. The method comprises receiving a three-dimensional digital dentition model, pairing a first and second three-dimensional digital tooth model of the tooth set, receiving an input defining a first transformation of the first three-dimensional digital tooth model, determining for the first transformation a first measure of an additional first shape-deforming, determining a second transformation of the second three-dimensional digital tooth model, determining for the second transformation a second measure of an additional second shape-deforming, determining a largest one of the two shape-deforming adjustments comprising a largest one of the two measures, adjusting the first and second three-dimensional digital tooth model.

### FIELD OF THE INVENTION

The invention relates to the field of dental technology. More particularly, the invention relates to a computer-implemented method, a computer device, and a computer program product for adjusting three-dimensional digital tooth models as well as to a manufacturing system comprising the computer device.

### BACKGROUND

In modern dental technology, computer-based approaches are used for configuring and manufacturing dental restorations. For a dental restoration, e.g., an arrangement of a plurality of three-dimensional digital tooth models may have to be generated and adjusted using a computer. Adjusting an arrangement of a plurality of three-dimensional digital tooth models for a dental restoration may be a challenging and work-intensive task.

### SUMMARY OF THE INVENTION

It is an objective to provide for a computer-implemented method, a computer device, and a computer program product for adjusting three-dimensional digital tooth models as well as for a manufacturing system comprising the computer device. The objectives underlying the invention are solved by the features of the independent claims.

In one aspect a computer-implemented method is disclosed for adjusting two or more three-dimensional digital tooth models of a tooth set of three-dimensional digital tooth models of a jaw. The tooth set is a tooth set for a dental restoration.

The method comprises receiving a three-dimensional digital dentition model comprising the tooth set and defining an arrangement of the three-dimensional digital tooth models of the tooth set. The three-dimensional digital dentition model further comprises a three-dimensional digital antagonistic model of one or more antagonistic structures of an antagonistic jaw and defines an arrangement of the three-dimensional digital antagonistic model relative to the arrangement of the three-dimensional digital tooth models of the tooth set.

A first three-dimensional digital tooth model of the tooth set descriptive of a first tooth is paired with a second three-dimensional digital tooth model of the tooth set descriptive of a second tooth being a contralateral counterpart tooth of the first tooth. An input defining a first transformation to be applied to the first three-dimensional digital tooth model is received.

For the first transformation a first measure of an additional first shape-deforming adjustment of a shape of the first three-dimensional digital tooth model is determined, which is required for preventing an intersection of the first three-dimensional digital tooth model with the three-dimensional digital antagonistic model due to the first transformation.

Using the input, a second transformation to be applied to the second three-dimensional digital tooth model is determined. The determining of the second transformation comprises a mapping of the first transformation to the second three-dimensional digital tooth model. For the second transformation a second measure of an additional second shape-deforming adjustment of a shape of the second three-dimensional digital tooth model is determined, which is required for preventing an intersection of the second three-dimensional digital tooth model with the three-dimensional digital antagonistic model due to the second transformation.

A largest one of the two shape-deforming adjustments is determined, which comprises a largest one of the two measures. The first three-dimensional digital tooth model and the second three-dimensional digital tooth model are adjusted. For example, the first three-dimensional digital tooth model and the second three-dimensional digital tooth model may be adjusted concurrently, i.e., simultaneously.

The adjusting comprises applying in combination the first transformation and the largest shape-deforming adjustment to the first three-dimensional digital tooth model. The first transformation and the largest shape-deforming adjustment are, e.g., applied concurrently in combination to the first three-dimensional digital tooth model. Furthermore, in combination the second transformation and the same largest shape-deforming adjustment are applied to the second three-dimensional digital tooth model. The second transformation and the same largest shape-deforming adjustment are, e.g., applied concurrently in combination to the second three-dimensional digital tooth model.

Example may have the beneficial effect that the first and second three-dimensional digital tooth model, when being applied with the first and second transformation, respectively, are not only applied with an individual minimum shape-deforming adjustment required for preventing intersections with the three-dimensional digital antagonistic model due to the respective transformation. The first and second three-dimensional digital tooth model may rather both be applied with the same largest shape-deforming adjustment. For one of the paired three-dimensional digital tooth models this largest shape-deforming adjustment may be a minimum adjustment required for preventing an intersection with the three-dimensional digital antagonistic model, while for the other three-dimensional digital tooth model this largest shape-deforming adjustment may be an adjustment, which is not or not to this extend required for preventing an intersection with the three-dimensional digital antagonistic model.

This may have the beneficial effect that intersections with three-dimensional digital antagonistic model may be effectively prevented for both three-dimensional digital tooth models, while at the same a symmetrical appearance of both three-dimensional digital tooth models may be maintained. By mapping the first transformation to the second three-dimensional digital tooth model for determining the second transformation of the second three-dimensional digital tooth model both three-dimensional digital tooth models may be transformed the same way. For example, the mapping may satisfy a mirror symmetry between the two three-dimensional digital tooth models with respect to a mirror plane arranged between the two, e.g., in a middle between the two. For example, the mapping may be configured such that an orientation, position, and/or size of the mapped first transformation, i.e., the second transformation, with respect to a second local coordinate frame of the second three-dimensional digital tooth model equals an orientation, position, and/or size of the first transformation being mapped with respect to a first local coordinate frame of the first three-dimensional digital tooth model. In case the two local coordinate frames satisfy a mirror symmetry with respect to a mirror plane, the transformations may also satisfy this mirror symmetry. In case the two local coordinate frames do not satisfy the mirror symmetry to the mirror plane, the transformations may not satisfy this mirror symmetry as well.

Mapping the first transformation to the second three-dimensional digital tooth model may result in a symmetrical appearance of the transformed three-dimensional digital tooth models. Starting with three-dimensional digital tooth models satisfying an exact global symmetry may result in transformed three-dimensional digital tooth models satisfying the exact global symmetry as well.

For applying the largest shape-deforming adjustment to the three-dimensional digital tooth model, for which it has not been determined, i.e., for which it is not required, the largest shape-deforming adjustment may be mapped analogously to the transformation. For example, the mapping may satisfy a mirror symmetry between the two three-dimensional digital tooth models with respect to a mirror plane arranged between the two, e.g., in a middle between the two. For example, the mapping may be configured such that an orientation, position, and/or size of the mapped shape-deforming adjustment with respect to a local coordinate frame of the three-dimensional digital tooth model, on which it is mapped, equals an orientation, position and/or size of the shape-deforming adjustment being mapped with respect to a local coordinate frame of the three-dimensional digital tooth model, from which it is mapped. In case the two local coordinate frames satisfy a mirror symmetry with respect to a mirror plane, the largest shape-deforming adjustment being applied may also satisfy this mirror symmetry. In case the two local coordinate frames do not satisfy the mirror symmetry to the mirror plane, the largest shape-deforming adjustment being applied may not satisfy this mirror symmetry as well.

By mapping the largest shape-deforming adjustment from one of the paired three-dimensional digital tooth models to the other, the shapes of both models may be deformed the same way. This may have the beneficial effect that the paired three-dimensional digital tooth models may preserve a symmetrical appearance although shape-deforming adjustments determined to be required for resolving antagonist intersections may be unsymmetric. Thereby, e.g., unsymmetric shapes due to shape-deforming adjustments to resolve antagonist intersections may be avoided.

The respective transformations and/or shape-deforming adjustments for the paired three-dimensional digital tooth models may, e.g., be determined for and/or applied to both three-dimensional digital tooth models simultaneously. For example, the respective transformations and/or shape-deforming adjustments for the paired three-dimensional digital tooth models may, e.g., be determined for and/or applied to both three-dimensional digital tooth models on-the-fly, while the user is providing input defining transformations of the first three-dimensional digital tooth model. For example, the user may select the first three-dimensional digital tooth model on a graphical user interface and apply a transformation to it, e.g., move the first three-dimensional digital tooth model. Simultaneously the paired second three-dimensional digital tooth model may be transformed the same way, e.g., be moved the same way. For example, the transformation of the paired second three-dimensional digital tooth model is applied relative to a second local coordinate frame of the second three-dimensional digital tooth model. Thus, transforming the same way, e.g., moving the same way, may refer to a transforming relative to the second local coordinate frame the same way as the first three-dimensional digital tooth model is transformed relative to a first local coordinate frame assigned to the first three-dimensional digital tooth model. Depending on the orientations of the first and second coordinate frames relative to each other and/or a direction of a transformation, e.g., movement, both three-dimensional digital tooth models may, e.g., be moved in the same direction or in opposite directions. While moving both three-dimensional digital tooth models a shape-deforming adjustment may be applied to both of same, e.g., simultaneously, as soon as at least one of the three-dimensional digital tooth models reaches an antagonistic structure defined by the three-dimensional digital antagonistic model, which requires a shape-deforming adjustment in order to prevent an intersection.

For example, the respective transformations and/or shape-deforming adjustments for the paired three-dimensional digital tooth models may, e.g., be determined for and/or applied to both three-dimensional digital tooth models repeatedly with rate of repetition, which is equal to or higher than a frame rate, which is used to display the three-dimensional digital tooth models on a display. Thus, the impression of a real-time transforming and/or adjusting of the paired three-dimensional digital tooth models may be implemented for the user.

In case for one of the paired three-dimensional digital tooth models, no shape-deforming adjustment is required for preventing an intersection with the three-dimensional digital antagonistic model relative due to the transformation, the measure being determined for this three-dimensional digital tooth model is zero. Thus, the other measure determined for the shape-deforming adjustment of the other one of the three-dimensional digital tooth models may be the largest one. As long as, there a shape-deforming adjustment is required for at least one of the paired three-dimensional digital tooth models, there will be a largest non-zero shape-deforming adjustment, which may be applied to both three-dimensional digital tooth models.

For example, the first measure of the additional first shape-deforming adjustment is different from the second measure of the additional second shape-deforming adjustment. Thus, there may be a single largest measure identifying a single largest shape-deforming adjustment. In case both measures are identical, both measures may be the largest one. Both shape-deforming adjustment may e.g., be identical. Thus, e.g., to each of the two three-dimensional digital tooth models the additional shape-deforming adjustment required for the respective three-dimensional digital tooth model may be applied. Thereby, the same largest shape-deforming adjustment may be applied to both three-dimensional digital tooth models.

For example, the measures of the shape-deforming adjustment may be determined section-wise. For example, for corresponding sections of a surface of the paired three-dimensional digital tooth models, which are deformed by a shape-deforming adjustment, a measure may be determined per section. Thus, also the determining of the largest measure and the largest shape-deforming adjustment as well as the applying of the largest shape-deforming adjustment may be performed section-wise. In different sections, different shape-deforming adjustments may be the largest one. For example, within a first section a shape-deforming adjustment of one of the three-dimensional digital tooth models may be the largest one, while for another second section a shape-deforming adjustment of the other one of the three-dimensional digital tooth models may be the largest one. Consequently, within the corresponding first sections of the paired three-dimensional digital tooth models and within the corresponding second sections of the paired three-dimensional digital tooth models different largest shape-deforming adjustments determined for different three-dimensional digital tooth models may be applied. One of the largest shape-deforming adjustments may be a shape-deforming adjustment determined for the first three-dimensional digital tooth model, while the other largest shape-deforming adjustment may be a shape-deforming adjustment determined for the second three-dimensional digital tooth model.

For example, the measures of the shape-deforming adjustment may be determined point-wise. For example, for corresponding surface points of a surface of the paired three-dimensional digital tooth models, which are deformed, i.e., displaced, by a shape-deforming adjustment, a measure may be determined quantifying the displacement per point. Thus, also the determining of the largest measure and the largest shape-deforming adjustment as well as the applying of the largest shape-deforming adjustment may be performed point-wise. For different surface points, different shape-deforming adjustments may be the largest one. For example, for a first surface point a shape-deforming adjustment of one of the three-dimensional digital tooth models may be the largest one, while for another second surface point a shape-deforming adjustment of the other one of the three-dimensional digital tooth models may be the largest one. Consequently, for the corresponding first surface points of the paired three-dimensional digital tooth models and for the corresponding second surface points of the paired three-dimensional digital tooth models different largest shape-deforming adjustments determined for different three-dimensional digital tooth models may be applied. One of the largest shape-deforming adjustments may be a shape-deforming adjustment determined for the first three-dimensional digital tooth model, while the other largest shape-deforming adjustment may be a shape-deforming adjustment determined for the second three-dimensional digital tooth model.

For example, the three-dimensional digital tooth models of a tooth set of three-dimensional digital tooth models are defined using meshes. A shape-deforming adjustment of a three-dimensional digital tooth model may, e.g., comprise an adjustment of at least one vertex, edge, and/or face of the mesh defining the respective three-dimensional digital tooth model. For example, the shape-deforming adjustment may comprise an adjustment of a position of at least one of the vertices relative to positions of other vertices comprised by the respective mesh.

The meshes may, e.g., be polygon meshes. A polygon mesh refers to a collection of vertices, edges and faces that defines a shape of a polyhedral object. The faces may, e.g., comprise triangles, quadrilaterals, or other n-gons.

For example, the three-dimensional digital tooth models of a tooth set of three-dimensional digital tooth models are defined using point clouds. A shape-deforming adjustment of a three-dimensional digital tooth model may, e.g., comprise an adjustment of at least one point of the point cloud defining the respective three-dimensional digital tooth model. For example, the shape-deforming adjustment may comprise an adjustment of a position of at least one of the points relative to positions of other points comprised by the respective point cloud.

A point cloud refers to a discrete set of data points in space, e.g., in three-dimensional space. The points may represent a three-dimensional shape or object. Each point position corresponds to a set of coordinates, e.g., a set of orthogonal coordinates. The set of orthogonal coordinates may, e.g., be Cartesian coordinates [X, Y, Z]. However, also other types of coordinates could be used instead, like, e.g., cylindrical polar coordinates or spherical coordinates.

For example, three-dimensional digital tooth models are each assigned with a local coordinate frame. These local coordinate frames are, e.g., orthogonal frames. For example, the first three-dimensional digital tooth model is assigned with a first local coordinate frame. For example, the paired second three-dimensional digital tooth model is assigned with a second local coordinate frame.

The local coordinate frames may, e.g., be Cartesian coordinate frames defined by an ordered triplet of coordinate axes. These coordinate axes, e.g., correspond to mesial, vestibular, and occlusal directions of the respective three-dimensional digital tooth model. For example, the origin of the local coordinate frame of a three-dimensional digital tooth model may be anchored at a spatial point of the respective digital tooth model, e.g., at the centroid.

A Cartesian coordinate frame for a three-dimensional space comprises an ordered triplet of lines, i.e., axes, which go through a common point, referred to as origin, and are pair-wise perpendicular. A Cartesian coordinate frame may, e.g., describe an orientation for each axis as well as a single unit of length for all three axes. The orientations of the three orthogonal axes of the local coordinate frame may, e.g., correspond to anatomical directions of a three-dimensional digital tooth model.

For example, a first axis is an axis oriented along a vestibular direction of the tooth model. A second axis may, e.g., be an axis oriented along a mesial direction of the tooth model and a third axis may, e.g., be an axis oriented along an occlusal direction of the tooth model.

The vestibular direction in case of posterior teeth may be equal to the buccal direction, while in case of anterior teeth the vestibular direction may be equal to the labial direction. For sake of simplicity, the term occlusal is used independently of the type of tooth described by the tooth model, i.e., posterior teeth as well as anterior teeth and generally refers to a coronal direction of the respective tooth. In other words, in case of anterior teeth it is used as a synonym for incisal. The mesial direction refers to a direction toward an anterior midline in a dental arch. Thus, for two adjacent incisors arranged on opposite sides of the anterior midline, the individual mesial directions may be opposite directions depending on the exact orientation of the respective tooth. This may, e.g., result in local coordinate frames with different handiness. For example, one of the local coordinate frames of two paired tooth models arranged on different hemispheres of the same jaw or on different jaws may be a left-handed coordinate frame, while the other local coordinate frame may be a right-handed coordinate frame.

The actual orientation of the axes of the local coordinate frames from a global point of view may differ depending on an orientation of the respective tooth models, they are assigned to. Since the local coordinate frames are assigned to the tooth models with a fixed relative orientation, their orientations may change with the orientations of the tooth models, they are assigned to. Therefore, they are referred to as local coordinate frames.

A position of a surface point pᵢ of a three-dimensional digital tooth model defining a geometric shape of a tooth may, e.g., in case of a mesh be defined by a position of a vertex Vᵢ, e.g., within the local coordinate frame of the respective three-dimensional digital tooth model. A shape-deforming adjustment of the position, i.e., a displacing of the surface point pᵢ may, e.g., be defined by a displaced position of the vertex Vᵢ, i.e., a displaced vertex position

$${V}_{i}^{def}$$

resulting from the shape-deforming adjustment, e.g., within the local coordinate frame of the respective three-dimensional digital tooth model. Along a reference direction [symbol] ᵢ, e.g., within the local coordinate frame of the respective three-dimensional digital tooth model, a measure of the displacing Δᵢ [symbol] ᵢ due to the shape-deforming adjustment may be Δᵢ and the displaced position

$${V}_{i}^{def}$$

of vertex Vᵢ resulting from the shape-deforming adjustment may be

$${V}_{i}^{def} = {V}_{i} + {Δ}_{i} {\vec{B}}_{i} .$$

The reference direction within the local coordinate frame of the respective three-dimensional digital tooth model may be defined by a unit vector [symbol] ᵢ in the respective direction.

For the first transformation an additional first shape-deforming adjustment of the shape of the first three-dimensional digital tooth model may be required for preventing an intersection of the first three-dimensional digital tooth model with the three-dimensional digital antagonistic model due to the first transformation. This first shape-deforming adjustment may result in a displacing of a first surface point p₁ of the first three-dimensional digital tooth model, e.g., be defined by a position of a first vertex V₁, along a first reference direction [symbol] ₁ by a first measure Δ₁ from the position of the first vertex V₁ to a position of the displaced first vertex resulting from the shape-deforming adjustment

$${V}_{1}^{def} = {V}_{1} + {Δ}_{1} {\vec{B}}_{1} .$$

For the second transformation an additional second shape-deforming adjustment of the shape of the second three-dimensional digital tooth model may be required for preventing an intersection of the second three-dimensional digital tooth model with the three-dimensional digital antagonistic model due to the second transformation. This second shape-deforming adjustment may result in a displacing of a second surface point p₂ of the second three-dimensional digital tooth model, e.g., be defined by a position of a second vertex V₂, along a second reference direction [symbol] ₂ by a second measure Δ₂ from the position of the second vertex V₂ to a position of the displaced second vertex resulting from the shape-deforming adjustment

$${V}_{2}^{def} = {V}_{2} + {Δ}_{2} {\vec{B}}_{2} .$$

For the two measures Δ₁ and Δ₂, a largest one of the two is determined, which identifies the largest one of the two shape-deforming adjustments Δ₁\vec{B}₁ and Δ₂\vec{B}₂. For example, the first measure Δ₁ may be the largest measure and therefore the first shape-deforming adjustment Δ₁\vec{B}₁ the largest adjustment. For example, the second measure Δ₂ may be the largest measure and therefore the second shape-deforming adjustment Δ₂\vec{B}₂ the largest adjustment.

The adjusting, e.g., a concurred adjusting, of the first and second three-dimensional digital tooth model may comprise an applying in combination the first transformation and the largest shape-deforming adjustment to the first three-dimensional digital tooth model. If Δ₁≥Δ₂, the largest shape-deforming adjustment applied to the first three-dimensional digital tooth model is Δ₁\vec{B}₁. If Δ₂>Δ₁, the largest shape-deforming adjustment applied to the first three-dimensional digital tooth model is Δ₂\vec{B}₁.

Furthermore, the second transformation and the same largest shape-deforming adjustment may be applied in combination to the second three-dimensional digital tooth model. If Δ₁>Δ₂, the largest shape-deforming adjustment applied to the second three-dimensional digital tooth model is Δ₁\vec{B}₂. Else, the largest shape-deforming adjustment applied to the second three-dimensional digital tooth model is Δ₂\vec{B}₂.

Using meshes for defining the surfaces of the three-dimensional digital tooth models, the shape-deforming adjustments may be defined by displacements of vertices relative to other vertices of the same mesh. A shape-deforming adjustment may comprise a plurality of vertices of a mesh being displaced. These displacements of vertices may be analyzed and used for determining the additional largest shape-deforming adjustment to be applied to the paired three-dimensional digital tooth models. For example, the shape-deforming adjustments may be analyzed and/or the largest shape-deforming adjustment may be determined and applied vertex-wise. These vertex-wise determined largest shape-deforming adjustments may origin from different three-dimensional digital tooth model of the paired three-dimensional digital tooth models. For example, some of the vertex-wise determined largest shape-deforming adjustments may be shape-deforming adjustments required by one of the three-dimensional digital tooth models of the paired three-dimensional digital tooth models to prevent intersections, while the other vertex-wise determined largest shape-deforming adjustments may be shape-deforming adjustments required by the other three-dimensional digital tooth model.

Concurrent adjusting may comprise different vertices of the same three-dimensional digital tooth model being adjusted with different largest shape-deforming adjustments, which may come from different three-dimensional digital tooth models of the paired three-dimensional digital tooth models. Different largest shape-deforming adjustments may come from a given three-dimensional digital tooth model or from a paired contralateral three-dimensional digital tooth model. A final result of shape-deforming adjustments applied to a given three-dimensional digital tooth model may thus, e.g., be a mixture of shape-deforming adjustments coming from both paired three-dimensional digital tooth models.

The definition of the first reference direction relative to the first local coordinate frame may equal the definition of the second reference direction relative to the second local coordinate frame. Even though from a global point of view, i.e., relative to a global coordinate frame, the two reference directions may in general be different. For example, the first reference direction may, e.g., be the vestibular direction of the first three-dimensional digital tooth model, e.g., [1, 0, 0]. For example, the second reference direction may, e.g., be the vestibular direction of the second three-dimensional digital tooth model, e.g., [1, 0, 0]. In case the two local coordinate frames are symmetric, e.g., mirror symmetric with respect to a mirror plane, the two reference directions may be symmetric as well. In case the two local coordinate frames are not symmetric, e.g., mirror symmetric with respect to a mirror plane, the two reference directions may also not be symmetric.

Working directly with reactions to antagonist structures, i.e., with shape-deforming adjustments for preventing intersections with the antagonistic structures defined by the three-dimensional digital antagonistic model, may save time, since a user may get direct feedback during applying a transformation to a three-dimensional digital tooth model. For example, shape-deforming adjustments may be implemented as instant anatomic tooth morphing (IATM) with active deformation in response to antagonistic structures. Instant anatomic tooth morphing may ensure that adjustments of geometric tooth shapes being calculated, e.g., in real-time, with respect to anatomical limitations defined by other tooth models, e.g., by antagonistic tooth models, antagonist scan models, and/or approximal tooth models. To fulfil imposed anatomical limitations defined by other tooth models, tooth deformation and/or tooth feature deformation may be applied to resolve one or more intersections of the selected and/or paired corresponding tooth model with one or more of the other tooth models defining the anatomical limitations. The instant anatomic tooth morphing may, e.g., be implemented using the local frame approach described herein. The local frame approach may, e.g., use a relative or an exact mirroring. The instant anatomic tooth morphing may, e.g., be implemented using an exact global symmetry enforced for the paired three-dimensional digital tooth models. The instant anatomic tooth morphing may, e.g., be implemented with or without grouping a plurality of three-dimensional digital tooth models of the tooth set as a chain-like assembly with a fixed relative arrangement of approximal three-dimensional digital tooth models.

Working with an active symmetry between the paired three-dimensional digital tooth models may increase an efficiency, since only one of the two paired three-dimensional digital tooth models, i.e. only one side of the jaw, may need to be considered. Only for the three-dimensional digital tooth model considered, i.e., the first three-dimensional digital tooth model a first transformation may be defined using an input. The mapped first transformation, i.e., the second transformation, as well as additional shape-deforming adjustments for both three-dimensional digital tooth may be determined and/or applied automatically using the respective input. Thus, e.g., two three-dimensional digital tooth models may be transformed and adjusted to antagonistic structures simultaneously. In addition, a symmetrical appearance of the paired three-dimensional digital tooth may be enforced.

Antagonistic structures, in particular natural antagonistic structures, may in general be asymmetric, i.e., may have asymmetric contacts with interacting three-dimensional digital tooth models. This may lead to asymmetric deformation results, when applying shape-deforming adjustments of the shape of the three-dimensional digital tooth models depending on the antagonistic structures, in order to prevent intersections of the respective three-dimensional digital tooth models with the three-dimensional digital antagonistic model defining the antagonistic structures. The three-dimensional digital antagonistic model may represent the natural antagonistic structures, e.g., asymmetric natural antagonistic structures.

Examples may take both shape-deforming adjustment results on both sides, i.e., for both three-dimensional digital tooth models, and perform a depth sorting along corresponding reference directions on both sides, i.e., for both three-dimensional digital tooth models. The reference directions may, e.g., be the directions along the vestibular axes of the respective teeth.

As a result, e.g., the surface points, like vertices, with the largest displacements may be identified on both sides, i.e., for both three-dimensional digital tooth models, and the respective displacements may be applied on both sides, i.e., to both three-dimensional digital tooth models.

It may be noted that the final result may take a combination of both shape-deforming adjustment results and propagate the respective combination of both, while still keeping the result symmetric. For different corresponding surface sections and/or surface points of the paired three-dimensional digital tooth models different ones of the shape-deforming adjustments may by larger. Thus, using, e.g., a section- and/or point-wise approach, the actual shape-deforming adjustment or adjustments being applied to the paired three-dimensional digital tooth models may be a combination of the first and second shape-deforming adjustment.

For example, the three-dimensional digital dentition model may be generated using a three-dimensional digital tissue model of a patient's intraoral tissue. This three-dimensional digital tissue model may, e.g., be descriptive of intraoral tissue of two jaws, i.e., maxilla and mandible. The three-dimensional digital tissue model may be generated using scan data of the intraoral tissue. The scan data may, e.g., be can data of a patient's oral cavity, an imprint of a patient's oral cavity and/or a positive of a patient's oral cavity generated using an imprint. For example, an intraoral optical scanner may be configured for scanning intraoral tissue within a patient's oral cavity comprising intraoral tissue. The intraoral tissue being scanned may, e.g., comprise hard and/or soft tissue. Hard tissue may, e.g., comprise teeth, while soft tissue may, e.g., comprise gingiva tissue. The intraoral tissue being scanned may, e.g., comprise one or more jaws of the patient, i.e., a mandible and/or a maxilla. A jaw being scanned may, e.g., be an edentulous jaw or a jaw comprising one or more teeth. Alternatively, an imprint of the intraoral tissue and/or a positive of the intraoral tissue generated using an imprint may be scanned.

The resulting three-dimensional digital tissue model may be used for providing the three-dimensional digital dentition model. Providing the three-dimensional digital dentition model may, e.g., comprise adding the tooth set for the dental restoration to the three-dimensional digital tissue model. The three-dimensional digital tooth models comprised by the tooth set may, e.g., be provided using three-dimensional digital tooth models in form of library teeth provided by a tooth library. The three-dimensional digital tooth models comprised by the tooth set may, e.g., be provided using scans of natural teeth or of physical tooth models. The three-dimensional digital tooth models comprised by the tooth set may, e.g., be generated from scratch.

For example, the applying of the largest shape-deforming adjustment comprises a mapping, e.g., a concurrent mapping, of the largest shape-deforming adjustment from an origin three-dimensional digital tooth model to a target three-dimensional digital tooth model. The origin three-dimensional digital tooth model may be the one of the first or second three-dimensional digital tooth model, for which the largest shape-deforming adjustment is determined. The target three-dimensional digital tooth model may be the other one of the first or second three-dimensional digital tooth model.

There may be an origin three-dimensional digital tooth, which is the one of the first and second three-dimensional digital tooth model, for which the lager shape-deforming adjustment was determined. This may be the first or the second three-dimensional digital tooth model depending on which deformation is the largest. The target three-dimensional digital tooth is the other one of the first and second three-dimensional digital tooth model.

Here, “origin” identifies the model for which the largest deformation is defined. “Target” identifies the model to which the largest deformation is mapped. For the target three-dimensional digital tooth model as the other one of the first and second three-dimensional digital tooth model a smaller shape-deforming adjustment may have been determined.

For example, the largest shape-deforming adjustment is the first shape-deforming adjustment determined for the first three-dimensional digital tooth model. The origin three-dimensional digital tooth model is the first three-dimensional digital tooth model, and the target three-dimensional digital tooth model is the second three-dimensional digital tooth model.

For example, the largest shape-deforming adjustment is the second shape-deforming adjustment determined for the second three-dimensional digital tooth model. The origin three-dimensional digital tooth model is the second three-dimensional digital tooth model, and the target three-dimensional digital tooth model is the first three-dimensional digital tooth model.

For example, the mapping of the largest shape-deforming adjustment comprises a mirroring of the largest shape-deforming adjustment onto the target three-dimensional digital tooth model at an adjustment mirror plane arranged between the origin and the target three-dimensional digital tooth model.

Thus, a symmetric implementation of the largest shape-deforming adjustment may be enabled. Starting with a symmetric first and second three-dimensional tooth model, the initial symmetry, e.g., a mirror symmetry with respect to the adjustment mirror plane, may be maintained, when applying the largest shape-deforming adjustment.

For example, the adjustment mirror plane is a sagittal plane. For example, the adjustment mirror plane divides the arrangement of the three-dimensional digital tooth models of the tooth set into two halves.

For example, the largest shape-deforming adjustment is mapped, e.g., concurrently mapped, to the target three-dimensional digital tooth model, such that a direction of the mapped largest shape-deforming adjustment relative to a target local coordinate frame of the target three-dimensional digital tooth model equals a direction of the largest shape-deforming adjustment relative to an origin local coordinate frame of the origin three-dimensional digital tooth model.

Thus, orientation, position, and/or size of the largest shape-deforming adjustment relative to a local coordinate frame may be maintained by the mapping. Thus, a symmetrical appearance of the three-dimensional digital tooth models may be supported. In case the local coordinate frames are configured symmetric to each other with respect, e.g., to a mirror plane, the mapped largest shape-deforming adjustment may be symmetric as well. In case the local coordinate frames are not configured symmetric to each other with respect, e.g., to a mirror plane, the mapped largest shape-deforming adjustment may also not be symmetric.

In case the local coordinate frames are aligned with anatomical directions of the three-dimensional digital tooth models, to which they are assigned, anatomical relations and/or dependencies of the largest shape-deforming adjustment may be preserved, when mapping the largest shape-deforming adjustment from one three-dimensional digital tooth model to the other.

For example, the first measure is a first distance of a displacing of a first surface section of the first three-dimensional digital tooth model along a first reference direction, which results from the first shape-deforming adjustment. For example, the second measure is a second distance of a displacing of a second surface section of the second three-dimensional digital tooth model along a second reference direction, which results from the second shape-deforming adjustment.

Using reference directions, the measures may be effectively quantified, enabling an efficient comparison of the shape-deforming adjustments. For example, the reference directions may correspond to anatomical directions of the three-dimensional digital tooth models, which may be defined by local coordinate frames of the respective three-dimensional digital tooth models. Thus, anatomical relations and/or dependencies of the shape-deforming adjustments may be effectively taken into account, when determining the measures.

For example, the first reference direction and the second reference direction are related by a mirror symmetry defined by the adjustment mirror plane. Example may enable a taking into account of the mirror symmetry, when determining the measures. Thus, symmetric measures may be used ensuring a comparability of the measures in dependency of the symmetry. This may be beneficial, when implementing the largest shape-deforming adjustment in asymmetry preserving way.

For example, a definition of the second reference direction relative to a second local coordinate frame of the second three-dimensional digital tooth model equals a definition of the first reference direction relative to a first local coordinate frame of the first three-dimensional digital tooth model.

Examples may take into account relations of the shape-deforming adjustments to the local coordinate frames of the three-dimensional digital tooth models, for which they are determined, when determining the measures. Thus, measures may be used, which describe the shape-deforming adjustments with respect to the local coordinate frames of the three-dimensional digital tooth models, for which they are determined. This may be beneficial, when implementing the largest shape-deforming adjustment taking using the local coordinate frames for mapping the largest shape-deforming adjustment between three-dimensional digital tooth models.

For example, the first reference direction is a vestibular direction of the first three-dimensional digital tooth model and the second reference direction is a vestibular direction of the second three-dimensional digital tooth model. For example, the first reference direction is an oral direction of the first three-dimensional digital tooth model and the second reference direction is an oral direction of the second three-dimensional digital tooth model. Since vestibular and oral directions are opposite direction, either one may be used. For example, both descriptions may only differ by opposite signs.

For example, the displacing of the first surface section of the first three-dimensional digital tooth model comprises a displacing of a first vertex of a first mesh defining the first surface section. For example, the displacing of the second surface section of the second three-dimensional digital tooth model comprises a displacing of a second vertex of a second mesh defining the second surface section.

Using meshes for defining the surfaces of the three-dimensional digital tooth models, the shape-deforming adjustments may be defined by displacements of vertices relative to other vertices of the same mesh. These displacements of vertices may be analyzed and used for determining the additional largest shape-deforming adjustment to be applied to the paired three-dimensional digital tooth models. For example, the shape-deforming adjustments may be analyzed and/or the largest shape-deforming adjustment may be determined and applied vertex-wise.

For example, the first measure is the first distance of the displacing of the first vertex. For example, the second measure is the second distance of the displacing of the second vertex. Examples enable a determining of the measures depending on displacements of vertices. For example, vertex-wise measures may thus be determined.

For example, the first and second vertex are corresponding vertices. The method may further comprise determining the corresponding first and second vertices. The determining of the corresponding first and second vertices may, e.g., comprise using one of the following: a ray intersection, a closest point determination, an interpolation, a three-dimensional coordinate transformation.

Example may enable a determining of corresponding vertices, which are displaced due to shape-deforming adjustments, and for which measures quantifying the respective displacements are determined and compared. These vertices may, e.g., symmetric to each other with respect to an exact global symmetry, e.g., a mirror symmetry. These vertices may, e.g., describe the same anatomical point for the respective three-dimensional tooth models.

For example, directions used for the respective computational techniques, e.g., directions of rays, may be determined by mapping a relative orientation of the respective directions, e.g., rays, from the local coordinate frame of one of the paired three-dimensional tooth models to an identical orientation relative to the local coordinate frame of the other one of the paired three-dimensional tooth models. In case the orientations of the local coordinate frames relative to each other are symmetric, also the orientation of the respective directions relative to each other may be symmetric. In case the orientations of the local coordinate frames relative to each other may not be symmetric, also the orientation of the respective directions relative to each other may not be symmetric from a global point of view.

Also, a determining of positions of one or more reference points, like vertices, as, e.g., used for a closest point determination, an interpolation, and/or a surface mapping algorithm may, e.g., comprise a determining of positions of one or more reference points on a surface of one of the paired three-dimensional tooth models and identify the same positions on a surface of the other one of the paired three-dimensional tooth models. The definition of a position with respect to the local coordinate frame of one of the paired three-dimensional tooth models may be identical with a definition of the corresponding position relative to the local coordinate frame of the other one of the paired three-dimensional tooth models.

For example, the displacing of the first surface section comprises a displacing of a first plurality of first vertices of the first mesh comprising the first vertex. For example, the displacing of the second surface section comprises a displacing of a second plurality of second vertices of the second mesh comprising the second vertex. The determining of the first measure may be executed vertex-wise per displaced first vertex of the first plurality of first vertices. The determining of the second measure may be executed vertex-wise per displaced second vertex of the second plurality of second vertices.

The determining of the largest shape-deforming adjustment may be executed vertex-wise and comprise a comparison of the vertex-wise determined first measure with the corresponding vertex-wise determined second measure. The applying, e.g., a concurrent applying, of the largest shape-deforming adjustment may be executed vertex-wise.

Examples may enable a vertex-wise determining and applying of the largest shape-deforming adjustment, which may, e.g., result in a mixed largest shape-deforming adjustment comprising shape adjustments required for different ones of the paired three-dimensional tooth models.

For example, the displacing of the first surface section of the first three-dimensional digital tooth model comprises a displacing of a first point of a first point cloud defining the first surface section. For example, the displacing of the second surface section of the second three-dimensional digital tooth model comprises a displacing of a second point of a second point cloud defining the second surface section.

Using point clouds for defining the surfaces of the three-dimensional digital tooth models, the shape-deforming adjustments may be defined by displacements of points relative to other points of the same point cloud. These displacements of points may be analyzed and used for determining the additional largest shape-deforming adjustment to be applied to the paired three-dimensional digital tooth models. For example, the shape-deforming adjustments may be analyzed and/or the largest shape-deforming adjustment may be determined and applied point-wise.

For example, the first measure is the first distance of the displacing of the first point. For example, the second measure is the second distance of the displacing of the second point. Examples enable a determining of the measures depending on displacements of points. For example, point-wise measures may thus be determined.

For example, the first and second point are corresponding points. The method may further comprise determining the corresponding first and second points. The determining of the corresponding first and second points may, e.g., comprise using one of the following: a ray intersection, a closest point determination, an interpolation, a three-dimensional coordinate transformation.

Example may enable a determining of corresponding points, which are displaced due to shape-deforming adjustments, and for which measures quantifying the respective displacements are determined and compared. These points may, e.g., symmetric to each other with respect to an exact global symmetry, e.g., a mirror symmetry. These points may, e.g., describe the same anatomical point for the respective three-dimensional tooth models.

For example, directions used for the respective computational techniques, e.g., directions of rays, may be determined by mapping a relative orientation of the respective directions, e.g., rays, from the local coordinate frame of one of the paired three-dimensional tooth models to an identical orientation relative to the local coordinate frame of the other one of the paired three-dimensional tooth models. In case the orientations of the local coordinate frames relative to each other are symmetric, also the orientation of the respective directions relative to each other may be symmetric. In case the orientations of the local coordinate frames relative to each other may not be symmetric, also the orientation of the respective directions relative to each other may not be symmetric from a global point of view.

Also, a determining of positions of one or more reference points, as, e.g., used for a closest point determination, an interpolation, and/or a surface mapping algorithm may, e.g., comprise a determining of positions of one or more reference points on a surface of one of the paired three-dimensional tooth models and identify the same positions on a surface of the other one of the paired three-dimensional tooth models. The definition of a position with respect to the local coordinate frame of one of the paired three-dimensional tooth models may be identical with a definition of the corresponding position relative to the local coordinate frame of the other one of the paired three-dimensional tooth models.

For example, the displacing of the first surface section comprises a displacing of a first plurality of first points of the first point cloud. For example, the displacing of the second surface section comprises a displacing of a second plurality of second points of the second point cloud. The determining of the first measure may be executed point-wise per displaced first point of the first plurality of first points. The determining of the second measure may be executed point-wise per displaced second point of the second plurality of second points.

The determining of the largest shape-deforming adjustment may be executed point-wise and comprise a comparison of the point-wise determined first measure with the corresponding point-wise determined second measure. The applying of the largest shape-deforming adjustment may be executed point-wise.

Examples may enable a point-wise determining and applying of the largest shape-deforming adjustment, which may, e.g., result in a mixed largest shape-deforming adjustment comprising shape adjustments required for different ones of the paired three-dimensional tooth models.

For example, the first transformation comprises one or more of the following: a translation, a rotation, a scaling, a deforming, an adding of tooth material, a removing of tooth material, a modification of a surface structure. For example, the second transformation comprises one or more of the following: a translation, a rotation, a scaling, a deforming, an adding of tooth material, a removing of tooth material, a modification of a surface structure.

A translation refers to a translational movement of the respective three-dimensional digital tooth model in a direction defined, e.g., relative to a local coordinate frame of the respective tooth model or a reference element of a symmetry being implemented, e.g., a mirror plane. A mapped translation may, e.g., be a replica of the aforementioned translational movement, but executed in a direction defined the same way relative to a local coordinate frame of the paired three-dimensional digital tooth model as the aforementioned translational movement has been defined relative to the local coordinate frame of the other three-dimensional digital tooth model. A mapped translation may, e.g., be a replica of the aforementioned translational movement, but executed in a direction defined in a symmetric way relative to the reference element of a symmetry being implemented.

A rotation refers to a rotational movement of the respective three-dimensional digital tooth model around a rotational axis arranged along a direction defined, e.g., relative to a local coordinate frame of the respective tooth model or a reference element of a symmetry being implemented, e.g., a mirror plane. A mapped rotation may, e.g., be a replica of the aforementioned rotational movement, but executed around an axis of rotation arranged along a direction defined the same way relative to a local coordinate frame of the paired three-dimensional digital tooth model as the aforementioned rotational movement has been defined relative to the local coordinate frame of the other three-dimensional digital tooth model. A mapped rotation may, e.g., be a replica of the aforementioned rotational movement, but executed around an axis of rotation arranged along a direction defined in a symmetric way relative to the reference element of a symmetry being implemented.

A scaling refers to a change of size of a three-dimensional digital tooth model. The change of size may be executed relative to a starting size of a three-dimensional digital tooth model. Thus, a three-dimensional digital tooth model may be scaled by X % of the starting size. The scaling may be an increasing or a decreasing of the starting size, e.g., by X %. The scaling factor may, e.g., be a scaling factor larger than one resulting in an increasing of the size of the respective three-dimensional digital tooth model. The scaling factor may, e.g., be a scaling factor lower than one resulting in a decreasing of the size of the respective three-dimensional digital tooth model.

A deforming refers to a modification of a geometric shape of the three-dimensional digital tooth model. This deforming may comprise modification in one or more directions defined relative to a local coordinate frame of the respective tooth model or a reference element of a symmetry being implemented, e.g., a mirror plane. A mapped deformation may be a replica of the aforementioned deformation, but executed in in one or more directions defined the same way relative to a local coordinate frame of the paired three-dimensional digital tooth model as the aforementioned deformation has been defined relative to the local coordinate frame of the other three-dimensional digital tooth model. A mapped deformation may, e.g., be a replica of the aforementioned deformation, but executed in a direction defined in a symmetric way relative to the reference element of a symmetry being implemented.

An adding and/or removing of tooth material, refers to a modification of a geometric shape of the three-dimensional digital tooth model by adding and/or removing of tooth material. This modification may comprise modifications in one or more directions defined relative to a local coordinate frame of the respective tooth model or a reference element of a symmetry being implemented, e.g., a mirror plane. A mapped adding and/or removing of tooth material may be a replica of the aforementioned adding and/or removing of tooth material, but executed in in one or more directions defined the same way relative to a local coordinate frame of the paired three-dimensional digital tooth model as the aforementioned adding and/or removing of tooth material has been defined relative to the local coordinate frame of the other three-dimensional digital tooth model. A mapped adding and/or removing of tooth material may, e.g., be a replica of the aforementioned adding and/or removing of tooth material, but executed in a direction defined in a symmetric way relative to the reference element of a symmetry being implemented.

A modification of a surface structure may modify an appearance of a surface structure of the respective three-dimensional digital tooth model. This modification may be executed on a surface section and/or structure defined relative to a local coordinate frame of the respective tooth model. A mapped modification of a surface structure may be a replica of the aforementioned modification, but executed on a surface section and/or structure defined the same way relative to a local coordinate frame of the paired three-dimensional digital tooth model the surface section and/or structure of the aforementioned modification has been defined relative to the local coordinate frame of the other three-dimensional digital tooth model. A modification of a surface structure may, e.g., be a replica of the aforementioned modification, but executed on a surface section and/or structure of the other three-dimensional digital tooth model defined in a symmetric way relative to the reference element of a symmetry being implemented.

For example, the mapping of the first transformation comprises a mirroring of the first transformation to the second three-dimensional digital tooth model at a transformation mirror plane arranged between the first and second three-dimensional digital tooth model.

Thus, a symmetric implementation of the first transformation may be enabled. Starting with a symmetric first and second three-dimensional tooth model, the initial symmetry, e.g., a mirror symmetry with respect to the transformation mirror plane, may be maintained, when mapping the first transformation to the second three-dimensional digital tooth model.

For example, the transformation mirror plane is the sagittal plane. For example, the transformation mirror plane divides the arrangement of the three-dimensional digital tooth models of the tooth set into two halves.

For example, the transformation mirror plane is identical with the adjustment mirror plane. Examples may ensure that transformations as well as additional shape-deforming adjustments both may satisfy the same mirror symmetry.

For example, the first transformation is mapped to the second three-dimensional digital tooth model, such that a directional definition of the resulting second transformation relative to the second local coordinate frame of the second three-dimensional digital tooth model equals a directional definition of the first transformation relative to the first local coordinate frame of the first three-dimensional digital tooth model.

Thus, orientation, position, and/or size of the first transformation relative to the first local coordinate frame may be maintained by the mapping. Thus, a symmetrical appearance of the three-dimensional digital tooth models may be supported. In case the local coordinate frames are configured symmetric to each other with respect, e.g., to a mirror plane, the mapped first transformation, i.e., the second transformation, may be symmetric as well. In case the local coordinate frames are not configured symmetric to each other with respect, e.g., to a mirror plane, the mapped first transformation, i.e., the second transformation, may also not be symmetric.

In case the local coordinate frames are aligned with anatomical directions of the three-dimensional digital tooth models, to which they are assigned, anatomical relations and/or dependencies of the first transformation may be preserved, when mapping the first transformation from the first three-dimensional digital tooth model to the second three-dimensional digital tooth model.

For example, the first and second three-dimensional digital tooth models are models of anterior teeth. For example, the first and second three-dimensional digital tooth models are models of one of the following tooth types: incisor, canine. By supporting and/or enforcing a symmetrical appearance of anterior teeth, a symmetrical appearance of a smile may be supported and/or enforced.

For example, the first and second three-dimensional digital tooth models are models of posterior teeth. For example, the first and second three-dimensional digital tooth models are models of one of the following tooth types: premolar, molar. Also, a symmetric appearance of posterior teeth may, e.g., be supported and/or enforced.

For example, the three-dimensional digital antagonistic model comprises an antagonistic tooth set of three-dimensional digital antagonistic tooth models of the antagonistic jaw arranged in an antagonistic arrangement.

For example, the tooth set and the antagonistic model, e.g., an antagonistic tooth set, of the three-dimensional digital dentition model are arranged in occlusion.

For example, the three-dimensional digital antagonistic model comprises an antagonist partial scan model. For example, the three-dimensional digital antagonistic model comprises an antagonist scan model. For example, the three-dimensional digital antagonistic model comprises a predefined antagonistic surface structure of the antagonistic jaw. The predefined antagonistic surface may, e.g., be provided using scan data of an antagonistic surface structure or using an artificially generated model of an antagonistic surface structure.

For example, the method further comprises generating a three-dimensional digital restoration model of the dental restoration comprising the tooth set with the adjusted first and second three-dimensional digital tooth models. Data for controlling a manufacturing of a physical dental restoration may be provided. The data may define the three-dimensional digital restoration model as a template for the physical dental restoration.

Examples may enable a manufacturing of the dental restoration using the adjusted first and second three-dimensional digital tooth models. For manufacturing the dental restoration or at least elements of the dental restoration, e.g., computer-controlled additive and/subtractive methods may be used.

For example, the method further comprises a manufacturing of the physical dental restoration using the data provided for controlling the manufacturing with the manufactured physical dental restoration being a physical copy of the template defined by the provided data.

Examples may enable a manufacturing of the dental restoration using the adjusted first and second three-dimensional digital tooth models.

For manufacturing the dental restoration or at least elements of the dental restoration, e.g., computer-controlled additive and/subtractive methods may be used. For example, the dental restoration being manufactured using at least one of the following: machining, three-dimensional printing, casting.

For example, the dental restoration is a set of crowns, in particular a set of implant-based crowns. For example, the dental restoration is a bridge. For example, the bridge is a partial bridge comprising a part of a dental arch. For example, the bridge is a complete bridge comprising a complete dental arch. For example, the dental restoration is a denture. For example, the denture is a partial denture comprising a part of a dental arch. For example, the denture is a full denture comprising a complete dental arch. For example, the denture is a removable denture. For example, the denture is a removable partial denture or a removable complete denture. For example, the denture is a fixed denture, i.e., implant-based denture. For example, the denture is a fixed partial denture or a fixed complete denture.

For example, the jaw is a mandible, while the antagonistic jaw is a maxilla. For example, the jaw is a maxilla, while the antagonistic jaw is a mandible.

For example, a trained machine learning module may be used for generating the three-dimensional digital dentition model with the tooth set. For generating the three-dimensional digital dentition model with the tooth set, the trained machine learning module may, e.g., use a three-dimensional digital tissue model of a patient's intraoral tissue. The three-dimensional digital tissue may, e.g., be provided using scan data of the dental cavity of the patient. The three-dimensional digital dentition model with the tooth set may be received as output from the trained machine learning module in response to providing the three-dimensional digital tissue model as input.

For example, the trained machine learning module may be provided. The trained machine learning module being provided may be trained to provide the three-dimensional digital dentition model with the tooth set as output in response to receiving the three-dimensional digital tissue model as input.

For example, the providing of the trained machine learning module may comprise providing a machine learning module to be trained. Training datasets may be provided for training the machine learning module to be trained. For example, each training dataset may comprise a three-dimensional digital training tissue model with a training tooth set as well as a three-dimensional digital training dentition model. The machine learning module to be trained may be trained to provide the three-dimensional digital training dentition models with the training tooth sets of the training datasets as an output in response to receiving the three-dimensional digital training tissue models of the respective training datasets as input.

For example, a trained machine learning module may be used for generating one or more of the three-dimensional digital tooth models of the tooth set of the three-dimensional dentition model. For generating the one or more three-dimensional digital tooth models, the trained machine learning module may, e.g., use a three-dimensional digital tissue model of a patient's intraoral tissue. The three-dimensional digital tissue may, e.g., be provided using scan data of the dental cavity of the patient. The one or more three-dimensional digital tooth models may be received as output from the trained machine learning module in response to providing the three-dimensional digital tissue model as input.

For example, the trained machine learning module may be provided. The trained machine learning module being provided may be trained to provide the one or more three-dimensional digital tooth models as output in response to receiving the three-dimensional digital tissue model as input.

For example, the providing of the trained machine learning module may comprise providing a machine learning module to be trained. Training datasets may be provided for training the machine learning module to be trained. For example, each training dataset may comprise a three-dimensional digital training tissue model as well as one or more three-dimensional digital training tooth models. The machine learning module to be trained may be trained to provide the one or more three-dimensional digital training tooth models of the training datasets as an output in response to receiving the three-dimensional digital training tissue models of the respective training datasets as input.

For example, a trained machine learning module may be used for assigning three-dimensional digital tooth models with local coordinate frames. A three-dimensional digital dentition model assigned with a local coordinate frame may be received as output from the trained machine learning module in response to providing the three-dimensional digital tooth model as input.

For example, the trained machine learning module may be provided. The trained machine learning module being provided may be trained to provide the three-dimensional digital dentition model assigned with a local coordinate frame as output in response to receiving the three-dimensional digital tooth model as input.

For example, the providing of the trained machine learning module may comprise providing a machine learning module to be trained. Training datasets may be provided for training the machine learning module to be trained. For example, each training dataset may comprise a three-dimensional digital training tooth model as well as a local coordinate frame assigned to the three-dimensional digital training tooth model. The machine learning module to be trained may be trained to provide the local coordinate frames assigned to the three-dimensional digital training tooth models as an output in response to receiving the three-dimensional digital training tooth models of the respective training datasets without local coordinate frames as input. The output may, e.g., be definitions of the assigned local coordinate frames or the three-dimensional digital training tooth models with the assigned local coordinate frames.

A machine learning module to be trained may, e.g., be an untrained machine learning module, a pre-trained machine learning module or a partially trained machine learning module. The machine learning module being trained may be an untrained machine learning module, which is trained from scratch. Alternatively, the machine learning module being trained may be a pre-trained or partially trained machine learning module. In general, it may not be necessary to start with an untrained machine learning module, e.g., in deep learning. For example, one may start with a pre-trained or partially trained machine learning module. The pre-trained or partially trained machine learning module may have been pre-trained or partially trained for the same or a similar task. Using a pre-trained or partially trained machine learning may, e.g., enable a faster training of the trained machine learning module to be trained, i.e., the training may converge faster. For example, transfer learning may be used for training a pre-trained or partially trained machine learning module. Transfer learning refers to a machine learning process, which rather than starting the learning process from scratch starts from patterns that have been previously learned, when solving a different problem. This way previous learnings may, e.g., be leveraged, avoiding to start from scratch. A pre-trained machine learning module is a machine learning module that was trained previously, e.g., on a large benchmark dataset to solve a problem similar to the one to be solved by the additional learning. In case of a pre-trained machine learning module a previous learning process has been completed successfully. A partially trained machine learning module is a machine learning module, which has been partially trained, i.e., the training process may not have been completed yet. A pre-trained or partially machine learning module may, e.g., be import and trained to be used for the purposes disclosed herein.

The term “machine learning” (ML) refers to a computer algorithm used to extract useful information from training data sets by building probabilistic models, which are referred to as machine learning modules or models, in an automated way. A machine learning module may also be referred to as a predictive model. Machine learning algorithms build a mathematical model based on sample data, known as “training data”, in order to make predictions or decisions without being explicitly programmed to perform the task. The machine learning module may be performed using a learning algorithm such as supervised or unsupervised learning. The machine learning module may be based on various techniques such as clustering, classification, linear regression, reinforcement, self-learning, support vector machines, neural networks, etc. A machine learning module may, e.g., be a data structure or program such as a neural network, in particular a convolutional neural network, a support vector machine, a decision tree, a Bayesian network etc. The machine learning module may be adapted to predict an unmeasured value, e.g., a three-dimensional digital dentition model with a tooth set, one or more three-dimensional digital tooth models, or a local coordinate frame assigned to a three-dimensional digital tooth model as output by the trained machine learning module. The trained machine learning module may predict the unmeasured value from other, known values, e.g., a three-dimensional digital tissue model or a three-dimensional digital tooth model without local coordinate frame as input. According to an example, the machine learning module may comprise a deep learning model.

For manufacturing the dental restoration or at least elements of the dental restoration, e.g., computer-controlled additive and/subtractive methods may be used. For example, the dental restoration being manufactured using at least one of the following: machining, three-dimensional printing, casting.

The dental restoration may, e.g., be a denture. For example, the dental restoration may, e.g., be a partial or a complete denture. A denture is a prosthetic device constructed to replace missing teeth and to be supported by surrounding soft and/or hard tissues of the oral cavity. The denture may, e.g., be a removable denture, e.g., a removable partial denture or a removable complete denture. Alternatively, the denture may, e.g., be a denture relying on bonding or clasping onto teeth or dental implants.

For example, the dental restoration may comprise one or more of the following: a veneer, a coping with coating, a crown, a bridge, a mockup, a waxup, a provisional.

A veneer is a layer of restoration material placed over a tooth, in order to cover one or more surfaces of the tooth. Veneers may, e.g., improve the aesthetics of a smile and/or protect the tooth's surface from damage. Indirect veneers are manufactured outside of a patient's oral cavity and then arranged on a tooth within the oral cavity. Direct veneers are built-up directly on a tooth inside a patient's oral cavity. The tooth may be prepared for receiving the veneer.

For example, two main types of restoration material may be used for manufacturing a veneer: composite and dental porcelain. A composite veneer may be directly placed on the tooth, i.e., built-up in the mouth of patient, or indirectly manufactured outside the mouth of the patient and later bonded to the tooth. In contrast, a porcelain veneer may only be indirectly manufactured outside the mouth of the patient. A full veneer crown, on the one hand, is dental restoration element that is configured to cover all the coronal tooth surfaces, i.e., the mesial, distal, facial, lingual, and occlusal surfaces. A laminate veneer, on the other hand, is a thin layer of restoration material that may, e.g., cover only a single surface of a tooth, e.g., a labial surface. A laminate veneer may generally be used for aesthetic purposes.

Coping with coating refers to a dental restoration element, which is directly built on the tooth to be restored. An underlying coping is arranged on the tooth. The coping is configured to replicate the performance of a natural tooth. On the coping a coating is applied, which is configured to replicate the natural aesthetics of the tooth to be restored. For example, a ceramic coating may be used. Using a coping with coating to restore a tooth may have the beneficial effect of providing a dental restoration element that combines both durability and natural aesthetics.

A crown is a dental restoration element in form of a dental cap. Such a crown may, e.g., be provided in form of a full coverage crown or a partial crown, like a ⅞ crown or a ¾ crown. Partial crowns, like ⅞ and ¾ crowns, are hybrids between an onlay and a full coverage crown. They are categorized based on an estimated degree of wall coverage of the walls of the prepared tooth, on which the respective crown is arranged. For example, a ¾ crown aims to cover three thirds of the walls of the tooth to be restored, e.g., three out of the four walls, e.g., with the buccal wall being spared. For example, a ⅞ crown aims to cover seven eights of the walls of the tooth to be restored. A full coverage crown completely caps or encircles a prepared tooth. A crown may, e.g., be required when a large cavity threatens the health of a tooth. A crown may be bonded to the tooth prepared for receiving the crown using a bonding material, e.g., a dental cement. A crown may be made from various materials, which may be fabricated using indirect methods, i.e., outside the patient's oral cavity. Crowns may be used to improve strength, to improve appearance of teeth and/or to halt deterioration.

A bridge is a dental restoration in form of a permanent appliance used to replace one or more missing teeth. A dental bridge comprises a plurality of artificial dental elements that are fused together, e.g., one or more artificial replacement teeth are definitively joined to adjacent teeth. A conventional bridge may be supported, e.g., by full coverage crowns, partial crowns, overlays, onlays or inlays on the abutment teeth. The abutment teeth require preparation and reduction to support the bridge.

A mockup prosthetic restoration composite is a composite to be arranged within a patient's mouth in order to visualize for the patient a result of a prosthetic restoration, before the actual prosthetic restoration is executed. Thus, the patient as well as a dentist may assess the expected esthetic and functional outcome of the prosthetic restoration. The final result to be expected may thus be visualized at an early stage of planning a prosthetic restoration. This approach may ensure that the patient as well as the dentist may have the same result to be achieved in mind and allows for potential adjustments to be made prior to the final restorations manufactured and applied, e.g., cemented.

A waxup prosthetic restoration refers to a prosthetic restoration made from laboratory wax. Such a waxup prosthetic restoration is used for acquiring information indicative of whether a specific prosthetic restoration is appropriate. A planned prosthetic restoration may be generated using from laboratory wax. The waxup prosthetic restoration may be used to test, whether the planned prosthetic restoration is appropriate. Using wax may have the beneficial effect, that the waxup prosthetic restoration may be easily adjusted to also test adjustments of the planned prosthetic restoration and/or adjusting the planned prosthetic restoration to requirements determined using the waxup prosthetic restoration.

A waxup model may, e.g., be used by a doctor and/or a practitioner for visualization purposes. Furthermore, it may, e.g., also be used for generating one or more in-mouth preparation guiding surfaces, e.g., using silicon imprints, where the doctor and/or practitioner may measure and/or visually gauge, whether a planned tooth reduction has been performed.

A provisional is a type of interim dental restoration designed to be a template for the final restoration. It is used to verify, e.g., a comfort in occlusion for the patient, esthetic parameters that satisfy the patient's and dentist's expected goals and/or phonetic evaluation for speech and airflow. Esthetic parameters may, e.g., comprise shape, midlines, smile line, embrasure shapes, and/or position of contacts. The phonetic evaluation for speech and airflow may ensure that no sibilance, whistlers, and/or lisp occur, and a clear articulation being enabled by the prosthetic restoration resembled by the provisional.

In another aspect, the invention relates to a computer program product for adjusting two or more three-dimensional digital tooth models of a tooth set of three-dimensional digital tooth models of a jaw. The tooth set is a tooth set for a dental restoration. The computer program product comprises a non-transitory computer readable storage medium having program instructions embodied therewith.

The program instructions are executable by a processing unit of a computer device to cause the computer device to receive a three-dimensional digital dentition model comprising the tooth set and defining an arrangement of the three-dimensional digital tooth models of the tooth set. The three-dimensional digital dentition model further comprises a three-dimensional digital antagonistic model of one or more antagonistic structures of an antagonistic jaw and defines an arrangement of the three-dimensional digital antagonistic model relative to the arrangement of the three-dimensional digital tooth models of the tooth set. A first three-dimensional digital tooth model of the tooth set descriptive of a first tooth is paired with a second three-dimensional digital tooth model of the tooth set descriptive of a second tooth being a contralateral counterpart tooth of the first tooth. An input defining a first transformation to be applied to the first three-dimensional digital tooth model is received.

For the first transformation a first measure of an additional first shape-deforming adjustment of a shape of the first three-dimensional digital tooth model is determined, which is required for preventing an intersection of the first three-dimensional digital tooth model with the three-dimensional digital antagonistic model due to the first transformation.

Using the input, a second transformation to be applied to the second three-dimensional digital tooth model is determined. The determining of the second transformation comprises a mapping of the first transformation to the second three-dimensional digital tooth model. For the second transformation a second measure of an additional second shape-deforming adjustment of a shape of the second three-dimensional digital tooth model is determined, which is required for preventing an intersection of the second three-dimensional digital tooth model with the three-dimensional digital antagonistic model due to the second transformation.

A largest one of the two shape-deforming adjustments is determined, which comprises a largest one of the two measures. The first three-dimensional digital tooth model and the second three-dimensional digital tooth model are adjusted. For example, the first three-dimensional digital tooth model and the second three-dimensional digital tooth model may be adjusted concurrently, i.e., simultaneously.

The adjusting comprises applying in combination the first transformation and the largest shape-deforming adjustment to the first three-dimensional digital tooth model. The first transformation and the largest shape-deforming adjustment are, e.g., applied concurrently in combination to the first three-dimensional digital tooth model. Furthermore, in combination the second transformation and the same largest shape-deforming adjustment are applied to the second three-dimensional digital tooth model. The second transformation and the largest shape-deforming adjustment are, e.g., applied concurrently in combination to the second three-dimensional digital tooth model.

The program instructions provided by the computer program product may, e.g., be executable by the processor of the computer device to cause the computer device to execute any of the aforementioned examples of the method for adjusting two or more three-dimensional digital tooth models of a tooth set of three-dimensional digital tooth models of a jaw.

In another aspect, the invention relates to a computer program for adjusting two or more three-dimensional digital tooth models of a tooth set of three-dimensional digital tooth models of a jaw. The tooth set is a tooth set for a dental restoration. The computer program comprises program instructions, which are executable by a processing unit of a computer device to cause the computer device to receive a three-dimensional digital dentition model comprising the tooth set and defining an arrangement of the three-dimensional digital tooth models of the tooth set. The three-dimensional digital dentition model further comprises a three-dimensional digital antagonistic model of one or more antagonistic structures of an antagonistic jaw and defines an arrangement of the three-dimensional digital antagonistic model relative to the arrangement of the three-dimensional digital tooth models of the tooth set. A first three-dimensional digital tooth model of the tooth set descriptive of a first tooth is paired with a second three-dimensional digital tooth model of the tooth set descriptive of a second tooth being a contralateral counterpart tooth of the first tooth. An input defining a first transformation to be applied to the first three-dimensional digital tooth model is received.

For the first transformation a first measure of an additional first shape-deforming adjustment of a shape of the first three-dimensional digital tooth model is determined, which is required for preventing an intersection of the first three-dimensional digital tooth model with the three-dimensional digital antagonistic model due to the first transformation.

Using the input, a second transformation to be applied to the second three-dimensional digital tooth model is determined. The determining of the second transformation comprises a mapping of the first transformation to the second three-dimensional digital tooth model. For the second transformation a second measure of an additional second shape-deforming adjustment of a shape of the second three-dimensional digital tooth model is determined, which is required for preventing an intersection of the second three-dimensional digital tooth model with the three-dimensional digital antagonistic model due to the second transformation.

A largest one of the two shape-deforming adjustments is determined, which comprises a largest one of the two measures. The first three-dimensional digital tooth model and the second three-dimensional digital tooth model are adjusted. For example, the first three-dimensional digital tooth model and the second three-dimensional digital tooth model may be adjusted concurrently, i.e., simultaneously.

The adjusting comprises applying in combination the first transformation and the largest shape-deforming adjustment to the first three-dimensional digital tooth model. The first transformation and the largest shape-deforming adjustment are, e.g., applied concurrently in combination to the first three-dimensional digital tooth model. Furthermore, in combination the second transformation and the same largest shape-deforming adjustment are applied to the second three-dimensional digital tooth model. The second transformation and the same largest shape-deforming adjustment are, e.g., applied concurrently in combination to the second three-dimensional digital tooth model.

The program instructions provided by the computer program may, e.g., be executable by the processor of the computer device to cause the computer device to execute any of the aforementioned examples of the method for adjusting two or more three-dimensional digital tooth models of a tooth set of three-dimensional digital tooth models of a jaw.

In another aspect, the invention relates to a computer device for adjusting two or more three-dimensional digital tooth models of a tooth set of three-dimensional digital tooth models of a jaw. The tooth set is a tooth set for a dental restoration. The computer device comprises a processing unit and a memory storing program instructions executable by the processing unit.

Execution of the program instructions by the processing unit causing the computer device to receive a three-dimensional digital dentition model comprising the tooth set and defining an arrangement of the three-dimensional digital tooth models of the tooth set. The three-dimensional digital dentition model further comprises a three-dimensional digital antagonistic model of one or more antagonistic structures of an antagonistic jaw and defines an arrangement of the three-dimensional digital antagonistic model relative to the arrangement of the three-dimensional digital tooth models of the tooth set.

A first three-dimensional digital tooth model of the tooth set descriptive of a first tooth is paired with a second three-dimensional digital tooth model of the tooth set descriptive of a second tooth being a contralateral counterpart tooth of the first tooth. An input defining a first transformation to be applied to the first three-dimensional digital tooth model is received.

For the first transformation a first measure of an additional first shape-deforming adjustment of a shape of the first three-dimensional digital tooth model is determined, which is required for preventing an intersection of the first three-dimensional digital tooth model with the three-dimensional digital antagonistic model due to the first transformation.

Using the input, a second transformation to be applied to the second three-dimensional digital tooth model is determined. The determining of the second transformation comprises a mapping of the first transformation to the second three-dimensional digital tooth model. For the second transformation a second measure of an additional second shape-deforming adjustment of a shape of the second three-dimensional digital tooth model is determined, which is required for preventing an intersection of the second three-dimensional digital tooth model with the three-dimensional digital antagonistic model due to the second transformation.

A largest one of the two shape-deforming adjustments is determined, which comprises a largest one of the two measures. The first three-dimensional digital tooth model and the second three-dimensional digital tooth model are adjusted. For example, the first three-dimensional digital tooth model and the second three-dimensional digital tooth model may be adjusted concurrently, i.e., simultaneously.

The adjusting comprises applying in combination the first transformation and the largest shape-deforming adjustment to the first three-dimensional digital tooth model. The first transformation and the largest shape-deforming adjustment are, e.g., applied concurrently in combination to the first three-dimensional digital tooth model. Furthermore, in combination the second transformation and the same largest shape-deforming adjustment are applied to the second three-dimensional digital tooth model. The second transformation and the same largest shape-deforming adjustment are, e.g., applied concurrently in combination to the second three-dimensional digital tooth model.

Execution of the program instructions by the processor of the computer device may, e.g., cause the computer device to execute any of the aforementioned examples of the method for adjusting two or more three-dimensional digital tooth models of a tooth set of three-dimensional digital tooth models of a jaw.

For example, the computer device is comprised by a manufacturing system. The computer device may be any of the aforementioned examples of a computer device. The manufacturing system may further comprise one or more manufacturing devices configured to manufacture the dental restoration.

Execution of the program instructions by the processing unit further causes the computer device to generate a three-dimensional digital restoration model of the dental restoration comprising the tooth set with the adjusted first and second three-dimensional digital tooth models. Data for controlling a manufacturing of a physical dental restoration is provided. The data defines the three-dimensional digital restoration model as a template for the physical dental restoration. The one or more manufacturing devices are controlled to manufacture the physical dental restauration using the data provided for controlling the manufacturing with the manufactured physical dental restauration being a physical copy of the template defined by the data provided.

For the manufacturing of the dental restoration, e.g., computer-controlled additive and/or subtractive methods may be used. For example, the dental restoration and/or elements of the dental restoration may be manufactured using one of the following: machining, 3D printing, casting.

For example, the one or more manufacturing devices of the manufacturing system may comprise one or more of the following: a machining device, a 3D printing device.

It is understood that one or more of the aforementioned examples may be combined as long as the combined examples are not mutually exclusive.

### BRIEF DESCRIPTION OF THE DRAWINGS

In the following, examples are described in greater detail making reference to the drawings in which:

FIG. 1 is a flowchart of an exemplary method for adjusting two or more three-dimensional digital tooth models of a tooth set;

FIG. 2 shows a schematic diagram illustrating exemplary shape-deforming adjustments of paired three-dimensional digital tooth models;

FIG. 3 shows an exemplary pair of a first and second three-dimensional digital tooth model;

FIG. 4 shows the exemplary pair of the first and second three-dimensional digital tooth model of FIG. 3 with individual shape-deforming adjustments applied;

FIG. 5 shows the exemplary pair of the first and second three-dimensional digital tooth model of FIG. 4 with a common largest shape-deforming adjustment applied;

FIG. 6 shows another exemplary pair of a first and second three-dimensional digital tooth model with a common largest shape-deforming adjustment applied;

FIG. 7 is a flowchart of an exemplary method for generating a dental restoration using an adjusted tooth set of three-dimensional digital tooth models for the dental restoration;

FIG. 8 shows an exemplary computer device for adjusting two or more three-dimensional digital tooth models of a tooth set for a dental restoration;

FIG. 9 shows an exemplary computer device for adjusting two or more three-dimensional digital tooth models of a tooth set for a dental restoration; and

FIG. 10 shows an exemplary system for manufacturing a dental restoration using an adjusted three-dimensional digital dentition model.

### DETAILED DESCRIPTION

In the following, similar elements are denoted by the same reference numerals.

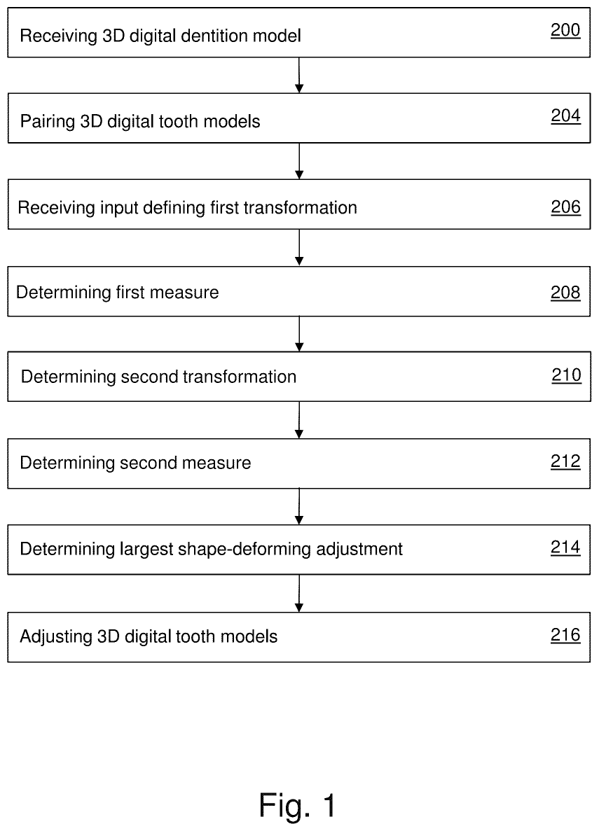

FIG. 1 shows a method for adjusting two or more three-dimensional digital tooth models of a tooth set of three-dimensional digital tooth models of a jaw. The tooth set is a tooth set for a dental restoration.

In block 200, a three-dimensional digital dentition model is received. The three-dimensional digital dentition model comprises the tooth set and defines an arrangement of the three-dimensional digital tooth models of the tooth set. The three-dimensional digital dentition model further comprises a three-dimensional digital antagonistic model of one or more antagonistic structures of an antagonistic jaw and defines an arrangement of the three-dimensional digital antagonistic model relative to the arrangement of the three-dimensional digital tooth models of the tooth set.

For example, the three-dimensional digital antagonistic model comprises an antagonistic tooth set of three-dimensional digital antagonistic tooth models of the antagonistic jaw arranged in an antagonistic arrangement. For example, the tooth set and the antagonistic model, e.g., an antagonistic tooth set, of the three-dimensional digital dentition model are arranged in occlusion. For example, the three-dimensional digital antagonistic model comprises an antagonist partial scan model. For example, the three-dimensional digital antagonistic model comprises an antagonist scan model. For example, the three-dimensional digital antagonistic model comprises a predefined antagonistic surface structure of the antagonistic jaw. The predefined antagonistic surface may, e.g., be provided using scan data of an antagonistic surface structure or using an artificially generated model of an antagonistic surface structure.

In block 204, a first three-dimensional digital tooth model of the tooth set descriptive of a first tooth is paired with a second three-dimensional digital tooth model of the tooth set descriptive of a second tooth being a contralateral counterpart tooth of the first tooth. In block 206, an input defining a first transformation to be applied to the first three-dimensional digital tooth model is received. The first transformation may, e.g., comprise one or more of the following: a translation, a rotation, a scaling, a deforming, an adding of tooth material, a removing of tooth material, a modification of a surface structure. In block 208, a first measure of an additional first shape-deforming adjustment of a shape of the first three-dimensional digital tooth model is determined for the first transformation. The additional first shape-deforming adjustment is required for preventing an intersection of the first three-dimensional digital tooth model with the three-dimensional digital antagonistic model due to the first transformation. For example, the first measure is a first distance of a displacing of a first surface section of the first three-dimensional digital tooth model along a first reference direction, which results from the first shape-deforming adjustment.

For example, the displacing of the first surface section of the first three-dimensional digital tooth model comprises a displacing of a first vertex of a first mesh defining the first surface section. For example, the first measure is the first distance of the displacing of the first vertex. For example, the displacing of the first surface section comprises a displacing of a first plurality of first vertices of the first mesh comprising the first vertex. The determining of the first measure may be executed vertex-wise per displaced first vertex of the first plurality of first vertices.

For example, the displacing of the first surface section of the first three-dimensional digital tooth model comprises a displacing of a first surface point of a first point cloud defining the first surface section. For example, the first measure is the first distance of the displacing of the first surface point.

For example, the displacing of the first surface section comprises a displacing of a first plurality of first surface points of the first point cloud. The determining of the first measure may be executed point-wise per displaced first point of the first plurality of first points.

In bock 210, a second transformation to be applied to the second three-dimensional digital tooth model is determined using the input. The determining of the second transformation comprises a mapping of the first transformation to the second three-dimensional digital tooth model. The second transformation may, e.g., comprise one or more of the following: a translation, a rotation, a scaling, a deforming, an adding of tooth material, a removing of tooth material, a modification of a surface structure. For example, the mapping of the first transformation comprises a mirroring of the first transformation to the second three-dimensional digital tooth model at a transformation mirror plane arranged between the first and second three-dimensional digital tooth model. The transformation mirror plane may, e.g., be a sagittal plane. It may, e.g., divide the arrangement of the three-dimensional digital tooth models of the tooth set into two halves. For example, the transformation mirror plane is identical with an adjustment mirror plane.

For example, the first transformation is mapped to the second three-dimensional digital tooth model, such that a directional definition of the resulting second transformation relative to the second local coordinate frame of the second three-dimensional digital tooth model equals a directional definition of the first transformation relative to the first local coordinate frame of the first three-dimensional digital tooth model.

In block 212, a second measure of an additional second shape-deforming adjustment of a shape of the second three-dimensional digital tooth model is determined for the second transformation. The additional second shape-deforming is required for preventing an intersection of the second three-dimensional digital tooth model with the three-dimensional digital antagonistic model due to the second transformation. For example, the second measure is a second distance of a displacing of a second surface section of the second three-dimensional digital tooth model along a second reference direction, which results from the second shape-deforming adjustment.

For example, the displacing of the second surface section of the second three-dimensional digital tooth model comprises a displacing of a second vertex of a second mesh defining the second surface section. For example, the second measure is the second distance of the displacing of the second vertex. For example, the first and second vertex are corresponding vertices. The method may further comprise determining the corresponding first and second vertices. The determining of the corresponding first and second vertices may comprise using one of the following: a ray intersection, a closest point determination, an interpolation, a three-dimensional coordinate transformation.

For example, the displacing of the second surface section comprises a displacing of a second plurality of second vertices of the second mesh comprising the second vertex. The determining of the second measure may be executed vertex-wise per displaced second vertex of the second plurality of second vertices.

For example, the displacing of the second surface section of the second three-dimensional digital tooth model comprises a displacing of a second surface point of a second point cloud defining the second surface section. For example, the second measure is the second distance of the displacing of the second surface point. For example, the first and second point are corresponding points. The method may further comprise determining the corresponding first and second points. The determining of the corresponding first and second points may comprise using one of the following: a ray intersection, a closest point determination, an interpolation, a three-dimensional coordinate transformation.

For example, the displacing of the second surface section comprises a displacing of a second plurality of second surface points of the second point cloud. The determining of the second measure may be executed point-wise per displaced second surface point of the second plurality of second surface points.

For example, the first reference direction and the second reference direction are related by a mirror symmetry defined by a mirror plane. The mirror plane may, e.g., be a sagittal plane. It may, e.g., divide the arrangement of the three-dimensional digital tooth models of the tooth set into two halves.

For example, a definition of the second reference direction relative to a second local coordinate frame of the second three-dimensional digital tooth model equals a definition of the first reference direction relative to a first local coordinate frame of the first three-dimensional digital tooth model. For example, the first reference direction is a vestibular direction of the first three-dimensional digital tooth model and the second reference direction is a vestibular direction of the second three-dimensional digital tooth model.

In block 214, a largest one of the two shape-deforming adjustments is determined, which comprises a largest one of the two measures.

For three-dimensional digital tooth models defined by meshes, the determining of the largest shape-deforming adjustment may, e.g., be executed vertex-wise and comprise a comparison of the vertex-wise determined first measure with the corresponding vertex-wise determined second measure. The applying, e.g., a concurrent applying, of the largest shape-deforming adjustment may be executed vertex-wise.

For three-dimensional digital tooth models defined by point clouds, the determining of the largest shape-deforming adjustment may be executed point-wise and comprise a comparison of the point-wise determined first measure with the corresponding point-wise determined second measure. The applying of the largest shape-deforming adjustment may be executed point-wise.

In block 216, the first three-dimensional digital tooth model and the second three-dimensional digital tooth model are adjusted. For example, the first three-dimensional digital tooth model and the second three-dimensional digital tooth model may be adjusted concurrently, i.e., simultaneously. The adjusting comprises applying in combination the first transformation and the largest shape-deforming adjustment to the first three-dimensional digital tooth model. The first transformation and the largest shape-deforming adjustment are, e.g., applied concurrently in combination to the first three-dimensional digital tooth model. Furthermore, in combination the second transformation and the same largest shape-deforming adjustment are applied to the second three-dimensional digital tooth model. The second transformation and the same largest shape-deforming adjustment are, e.g., applied concurrently in combination to the second three-dimensional digital tooth model.

The applying of the largest shape-deforming adjustment comprises, e.g., a mapping, e.g., a concurrent mapping, of the largest shape-deforming adjustment from an origin three-dimensional digital tooth model to a target three-dimensional digital tooth model. The origin three-dimensional digital tooth model may be the one of the first or second three-dimensional digital tooth model, for which the largest shape-deforming adjustment is determined. The target three-dimensional digital tooth model may be the other one of the first or second three-dimensional digital tooth model.

For example, the largest shape-deforming adjustment is the first shape-deforming adjustment determined for the first three-dimensional digital tooth model. The origin three-dimensional digital tooth model is the first three-dimensional digital tooth model, and the target three-dimensional digital tooth model is the second three-dimensional digital tooth model. For example, the largest shape-deforming adjustment is the second shape-deforming adjustment determined for the second three-dimensional digital tooth model. The origin three-dimensional digital tooth model is the second three-dimensional digital tooth model, and the target three-dimensional digital tooth model is the first three-dimensional digital tooth model.

For example, the mapping of the largest shape-deforming adjustment comprises a mirroring of the largest shape-deforming adjustment onto the target three-dimensional digital tooth model at an adjustment mirror plane arranged between the origin and the target three-dimensional digital tooth model. The adjustment mirror plane may, e.g., be a sagittal plane. It may, e.g., divide the arrangement of the three-dimensional digital tooth models of the tooth set into two halves.

For example, the largest shape-deforming adjustment is mapped, e.g., concurrently mapped, to the target three-dimensional digital tooth model, such that a direction of the mapped largest shape-deforming adjustment relative to a target local coordinate frame of the target three-dimensional digital tooth model equals a direction of the largest shape-deforming adjustment relative to an origin local coordinate frame of the origin three-dimensional digital tooth model.

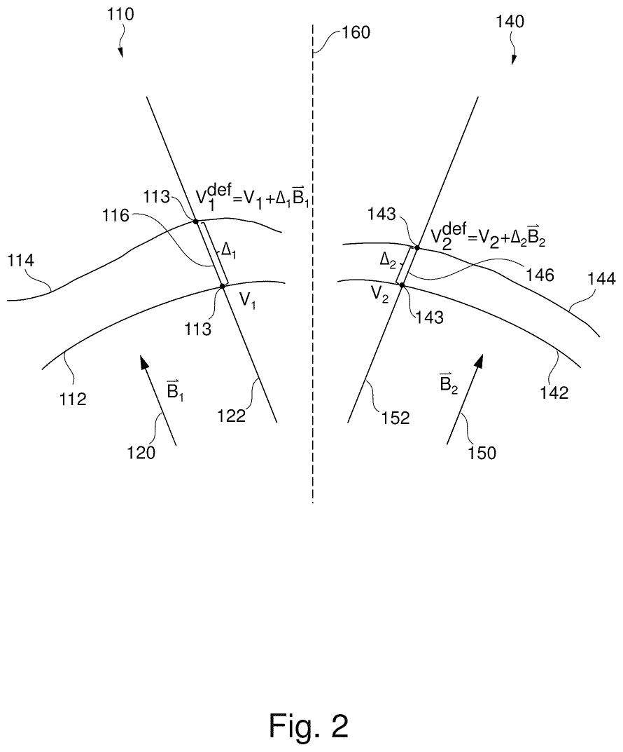

FIG. 2 shows a schematic diagram illustrating shape-deforming adjustments of paired three-dimensional digital tooth models 110, 140. A first three-dimensional digital tooth model 110 comprises a first surface section 112 with a first surface point 113, e.g., a vertex in case of a mesh. A position of the first surface point 113 may, e.g., be defined by a position of a first vertex V₁, e.g., within a local coordinate frame of the respective three-dimensional digital tooth model 110.

A first transformation of the first three-dimensional digital tooth model 110 may require an additional first shape-deforming adjustment of a shape of the first three-dimensional digital tooth model 110 and thus of the first surface section 112 for preventing an intersection of the first three-dimensional digital tooth model 110 with a three-dimensional digital antagonistic model due to the first transformation. This first shape-deforming adjustment may result in a deformed first surface section 114 comprising the first surface point 113, which has been displaced due to the shape-deforming adjustment. Along a first reference direction [symbol] ₁, e.g., within the local coordinate frame of the respective first three-dimensional digital tooth model 110, a measure 116 of the displacing due to the first shape-deforming adjustment may be Δ₁. This reference direction is further indicated by line 122 through surface point 113, i.e. the first vertex V₁, which extends parallel to the reference direction \vec{B}₁. The resulting displaced position

$${V}_{1}^{def}$$

of first surface point 113 of the deformed first surface section 114, i.e. of the first displaced vertex, may be

$${V}_{1}^{def} = {V}_{1} + {Δ}_{1} {\vec{B}}_{1} .$$

The first reference direction 120 within the local coordinate frame of the respective three-dimensional digital tooth model may, e.g., be defined by a first unit vector \vec{B}₁ in the respective direction. The direction may, e.g., be a vestibular direction of the first three-dimensional digital tooth model 110.

A second three-dimensional digital tooth model 140 comprises a second surface section 142 with a second surface point 143, e.g., a vertex in case of a mesh. A position of the second surface point 143 may be defined by a position of a second vertex V₂, e.g., within a local coordinate frame of the respective three-dimensional digital tooth model 140.

A second transformation of the second three-dimensional digital tooth model 140 may require an additional second shape-deforming adjustment of a shape of the second three-dimensional digital tooth model 140 and thus of the second surface section 142 for preventing an intersection of the second three-dimensional digital tooth model 140 with a three-dimensional digital antagonistic model due to the second transformation. The second transformation may result from a mapping of the first transformation to the second three-dimensional digital tooth model 140. The second shape-deforming adjustment may result in a deformed second surface section 144 comprising the second surface point 143, which has been displaced due to the shape-deforming adjustment. Along a second reference direction \vec{B}₂, e.g., within the local coordinate frame of the respective second three-dimensional digital tooth model 140, a measure 146 of the displacing due to the second shape-deforming adjustment may be Δ₂. This reference direction is further indicated by line 152 through surface point 143, i.e. the second vertex V₂, which extends parallel to the reference direction \vec{B}₁. The resulting displaced position

$${V}_{2}^{def}$$

of second surface point 143 of the deformed second surface section 144 may be

$${V}_{2}^{def} = {V}_{2} + {Δ}_{2} {\vec{B}}_{2} .$$

The second reference direction 150 within the local coordinate frame of the respective three-dimensional digital tooth model may, e.g., be defined by a second unit vector [symbol] ₂ in the respective direction. The direction may, e.g., be a vestibular direction of the second three-dimensional digital tooth model 140.

For the two measures Δ₁ and Δ₂, a largest one of the two is determined, which identifies the largest one of the two shape-deforming adjustments Δ₁\vec{B}₁ and Δ₂\vec{B}₂. For example, the first measure Δ₁ may be the largest measure and therefore the first shape-deforming adjustment Δ₁\vec{B}₁ the largest adjustment. For example, the second measure Δ₂ may be the largest measure and therefore the second shape-deforming adjustment Δ₂\vec{B}₂ the largest adjustment.

The adjusting of the first and second three-dimensional digital tooth model 110, 140 may comprise an applying, e.g., concurrently, in combination the first transformation and the largest shape-deforming adjustment to the first three-dimensional digital tooth model 110. If Δ₁≥Δ₂, the largest shape-deforming adjustment applied to the first three-dimensional digital tooth model 110 is Δ₁\vec{B}₁ with the new position of the displaced first vertex resulting from the shape-deforming adjustment

$${V}_{1}^{def} = {V}_{1} + {Δ}_{1} {\vec{B}}_{1} .$$

If Δ₂>Δ₁, the largest shape-deforming adjustment applied to the first three-dimensional digital tooth model 110 is Δ₂\vec{B}₁ with the new position of the displaced first vertex resulting from the shape-deforming adjustment

$${V}_{1}^{def} = {V}_{1} + {Δ}_{2} {\vec{B}}_{1} .$$

Furthermore, the second transformation and the same largest shape-deforming adjustment may be applied in combination to the second three-dimensional digital tooth model 140. If Δ₁>Δ₂, the largest shape-deforming adjustment applied to the second three-dimensional digital tooth model 140 is Δ₁\vec{B}₂ with the new position of the displaced second vertex resulting from the shape-deforming adjustment

$${V}_{2}^{def} = {V}_{2} + {Δ}_{1} {\vec{B}}_{2} .$$

Else, largest shape-deforming adjustment applied to the second three-dimensional digital tooth model 140 is Δ₂ [symbol] ₂ with the new position of the displaced second vertex resulting from the shape-deforming adjustment

$${V}_{2}^{def} = {V}_{2} + {Δ}_{2} {\vec{B}}_{2} .$$

The definition of the first reference direction [symbol] ₁ 120 relative to the first local coordinate frame of the first three-dimensional digital tooth model 110 may equal the definition of the second reference direction [symbol] ₂ 150 relative to the second local coordinate frame of the second three-dimensional digital tooth model 140. Even though from a global point of view, i.e., relative to a global coordinate frame, the two reference directions 120, 150 may in general be different. For example, the first reference direction may, e.g., be the vestibular direction of the first three-dimensional digital tooth model 120, e.g., [1, 0, 0]. For example, the second reference direction 150 may, e.g., be the vestibular direction of the second three-dimensional digital tooth model 140, e.g., [1, 0, 0].

For example, the first and second reference direction 120, 150 may be related by a mirror symmetry defined by a mirror plane 160. The mirror plane 160 may, e.g., be a sagittal plane. It may, e.g., divide the arrangement of the three-dimensional digital tooth models of the tooth set into two halves. For example, the local coordinate frames of the first and second three-dimensional tooth model 110, 140 may be related by mirror symmetry defined by a mirror plane 160, resulting in the mirror symmetry between the first and the second reference direction 120, 150. For example, positions, orientations, and/or shapes of the paired three-dimensional tooth models 110, 140 may be related by mirror symmetry defined by a mirror plane 160.

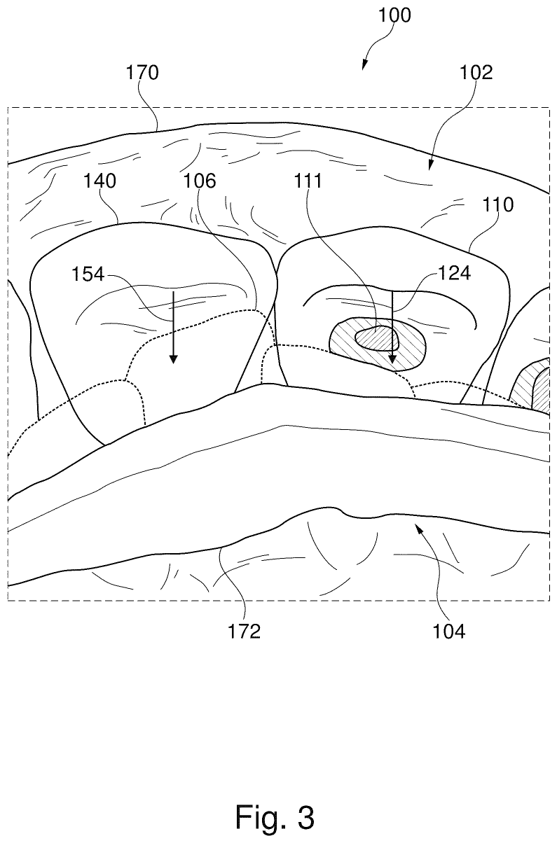

FIG. 3 shows an exemplary pair of a first and second three-dimensional digital tooth model 110, 140. A three-dimensional digital dentition model 100 comprising a tooth set 102 with a plurality of three-dimensional digital tooth models of a jaw 170. The three-dimensional digital dentition model further 100 further comprises a three-dimensional digital antagonistic model 104 of one or more antagonistic structures 106 of an antagonistic jaw 172. The three-dimensional digital dentition model further 100 further defines an arrangement of the three-dimensional digital antagonistic model 104 relative to the tooth set 102, i.e., to the arrangement of the three-dimensional digital tooth models of the tooth set 102.

The first three-dimensional digital tooth model 110 of the tooth set 102 descriptive of a first tooth is paired with a second three-dimensional digital tooth model 140 of the tooth set 102 descriptive of a second tooth being a contralateral counterpart tooth of the first tooth. In the example according to FIG. 3, the first three-dimensional digital tooth model 110 is a model of an incisor 21 according to FDI notation, while the second three-dimensional digital tooth model 140 is a model of an incisor 11 according to FDI notation.

Paired contralateral counterpart teeth may be pairs of any contralateral counterpart teeth. For example, the paired three-dimensional digital tooth models may be the other first incisors, i.e., 31 and 41 according to FDI notation. For example, the paired three-dimensional digital tooth models may be the second incisors, i.e., 12 and 22 or 32 and 42 according to FDI notation. For example, the paired three-dimensional digital tooth models may be canines, i.e., 13 and 23 or 33 and 43 according to FDI notation. For example, the paired three-dimensional digital tooth models may be the first premolars, i.e., 14 and 24 or 34 and 44 according to FDI notation. For example, the paired three-dimensional digital tooth models may be the second premolars, i.e., 15 and 25 or 35 and 45 according to FDI notation. For example, the paired three-dimensional digital tooth models may be the first molars, i.e., 16 and 26 or 36 and 46 according to FDI notation. For example, the paired three-dimensional digital tooth models may be the second molars, i.e., 17 and 27 or 37 and 47 according to FDI notation. For example, the paired three-dimensional digital tooth models may be the third molars, i.e., 18 and 28 or 38 and 48 according to FDI notation.

FIG. 3 shows, e.g., starting positions of the first and second three-dimensional digital tooth model 110, 140 before an input defining a first transformation to be applied to the first three-dimensional digital tooth model 110 is received. The first transformation may, e.g., comprise a translation, i.e., a moving of the first three-dimensional digital tooth model 110 in an oral direction 124 of the first three-dimensional digital tooth model 110. The translation may, e.g., be applied to adjust a position of the first three-dimensional digital tooth model 110, in order to reduce an intersection 111 of the first three-dimensional digital tooth model 110 with a dental restoration, on which the first three-dimensional digital tooth model 110 is arranged. Such a translation may require an additional first shape-deforming adjustment of a shape of the first three-dimensional digital tooth model 110 for preventing an intersection of the first three-dimensional digital tooth model 110 with the three-dimensional digital antagonistic model 104 due to the first transformation.

Using the input, a second transformation to be applied to the second three-dimensional digital tooth model 140 may be determined. The second transformation may comprise the first transformation mapped to the second three-dimensional digital tooth model 140, e.g., a translation of the second three-dimensional digital tooth model 140 in an oral direction 154 of the second three-dimensional digital tooth model 140. Such a translation may require an additional second shape-deforming adjustment of a shape of the second three-dimensional digital tooth model 140 for preventing an intersection of the second three-dimensional digital tooth model 140 with the three-dimensional digital antagonistic model 104 due to the second transformation.

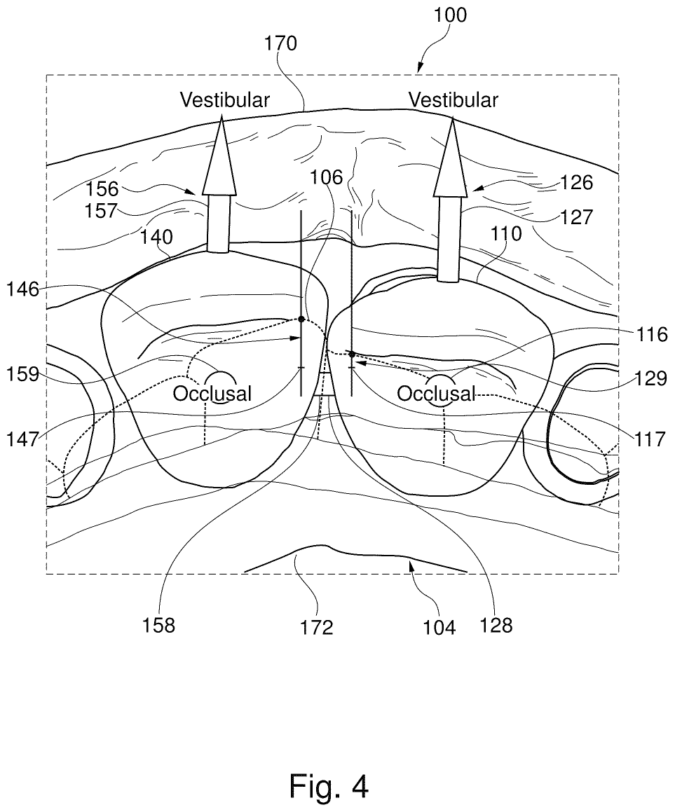

FIG. 4 shows the pair of the first and second three-dimensional digital tooth model 110, 140 of FIG. 3 with the additional first shape-deforming adjustment of the first three-dimensional digital tooth model 110 required for preventing an intersection with the three-dimensional digital antagonistic model 104 and additional second shape-deforming adjustment of the second three-dimensional digital tooth model 140 required for preventing an intersection with the three-dimensional digital antagonistic model 104.

Starting from the starting positions of the first and second three-dimensional digital tooth model 110, 140 shown in FIG. 3, the first and second three-dimensional digital tooth model 110, 140 have been moved in oral direction towards the three-dimensional digital antagonistic model 104 with the antagonistic structures 106. These movements, i.e., translations, are the result of the first transformation applied to the first three-dimensional digital tooth model 110 and the second transformation applied to the second three-dimensional digital tooth model 140.

The first translation of the first three-dimensional digital tooth model 110 requires an additional first shape-deforming adjustment of the shape of the first three-dimensional digital tooth model 110 to prevent an intersection of the first three-dimensional digital tooth model 110 with the three-dimensional digital antagonistic model 104 due to the respective translation. The second translation of the second three-dimensional digital tooth model 140 requires an additional second shape-deforming adjustment of the shape of the second three-dimensional digital tooth model 140 to prevent an intersection of the second three-dimensional digital tooth model 140 with the three-dimensional digital antagonistic model 104 due to the respective translation.

The first translation of the first three-dimensional digital tooth model 110 would have resulted in an end position 117 of the surface, e.g., the oral surface, of first three-dimensional digital tooth model 110. However, this would have resulted in an intersection with the antagonistic structure 106 defined by the three-dimensional digital antagonistic model 104. In order to prevent this intersection, the respective surface section is displaced using the additional first shape-deforming adjustment of the shape of the first three-dimensional digital tooth model 110. This first shape-deforming adjustment comprises a displacing of the respective oral surface section along the vestibular direction as a reference direction by a first distance. This first distance is a first measure 116 of the first shape-deforming adjustment.

The second translation of the second three-dimensional digital tooth model 140 would have resulted in an end position 147 of the surface, e.g., the oral surface, of second three-dimensional digital tooth model 140. However, this would have resulted in an intersection with the antagonistic structure 106 defined by the three-dimensional digital antagonistic model 104. In order to prevent this intersection, the respective surface section is displaced using the additional second shape-deforming adjustment of the shape of the second three-dimensional digital tooth model 140. This second shape-deforming adjustment comprises a displacing of the respective oral surface section along the vestibular direction as a reference direction by a second distance. This second distance is a second measure 146 of the second shape-deforming adjustment.

In the example of FIG. 4, the second measure 146 is larger than the first measure 116, i.e., the shape of the first three-dimensional digital tooth model 110 has to be deformed less than the shape of the second three-dimensional digital tooth model 140, in order to prevent intersection with the three-dimensional digital antagonistic model 104. This is due to the fact, that the antagonistic structure 106 defined by the three-dimensional digital antagonistic model 104 is asymmetric with respect to the first and second three-dimensional digital tooth model 110, 140. A section of the antagonistic structure 106 antagonistic to the second three-dimensional digital tooth model 110, e.g., an antagonist of the respective three-dimensional digital tooth model, may, e.g., protrude further in vestibular direction than a section of the antagonistic structure 106 antagonistic to the first three-dimensional digital tooth model 110, e.g., an antagonist of the respective three-dimensional digital tooth model.

However, these different requirements for shape-deforming adjustments may result in an asymmetrical appearance of the paired first and second three-dimensional digital tooth model 110, 140, even in case the first and second transformations applied to the three-dimensional digital tooth model, respectively, are symmetric. In order to be able to ensure a more symmetrical appearance of the adjusted three-dimensional digital tooth models 110, 140, a largest one of the two shape-deforming adjustments comprising a largest one of the two measures is determined. In the example, shown in FIG. 4, the second shape-deforming adjustment with the second measure 146 is the largest shape-deforming adjustment. Thus, this largest shape-deforming adjustment may be applied, e.g., concurrently, to both the three-dimensional digital tooth models 110, 140, rather than applying different shape-deforming adjustments as shown in FIG. 4. Applying the same largest shape-deforming adjustment to both three-dimensional digital tooth models 110, 140, may on the one hand side ensure that none of the three-dimensional digital tooth models 110, 140 has intersections with the antagonistic structure 106 defined by the three-dimensional digital antagonistic model 104 due to the transformations and on the other hand side ensure a symmetrical appearance of the paired three-dimensional digital tooth models 110, 140.

Both three-dimensional digital tooth models 110, 140 may, each be assigned with a local coordinate frame 126, 156. The local coordinate frames 126, 156 may, e.g., be Cartesian coordinate frames. A Cartesian coordinate frame or system for a three-dimensional space comprises an ordered triplet of axes, which go through a common point, referred to as the origin, and are pair-wise perpendicular. A Cartesian coordinate frame may describe an orientation for each axis as well as a single unit of length for all three axes. In the example shown, the orientations of the local coordinate frames are anatomical directions of the three-dimensional digital tooth models, to which they are assigned. For example, a first axis 127, 157 is an axis oriented along a vestibular direction of the first three-dimensional digital tooth model 110 and the second three-dimensional digital tooth model 140, respectively. A second axis 128, 158 may, e.g., be an axis oriented along a mesial direction of the first three-dimensional digital tooth model 110 and the second three-dimensional digital tooth model 140, respectively. A third axis 129, 159 may, e.g., be an axis oriented along an occlusal direction of the first three-dimensional digital tooth model 110 and the second three-dimensional digital tooth model 140, respectively. Since the two three-dimensional digital tooth models 110, 140 are contralateral counterpart teeth arranged on different hemispheres of the same jaw 170, i.e., on different sides of the midline, the mesial directions of the three-dimensional digital tooth models 110, 140 are opposite directions. The first local coordinate frame 126 of the first three-dimensional digital tooth models 110 is, e.g., a right-handed coordinate frame, while the second local coordinate frame 156 of the second three-dimensional digital tooth models 140 is, e.g., a left-handed coordinate frame.

For mapping the first transformation, i.e., first translation, to the second three-dimensional digital tooth model 140, the local coordinate frames 126, 156 assigned to the three-dimensional digital tooth models 110, 140 may be used as references for determining directions. The first transformation may be mapped to the second three-dimensional digital tooth model 140, such that a directional definition of the resulting second transformation relative to the second local coordinate frame 156 of the second three-dimensional digital tooth model 140 equals a directional definition of the first transformation relative to the first local coordinate frame 126 of the first three-dimensional digital tooth model 110. For example, a first transformation of the first three-dimensional digital tooth model 110 in the vestibular direction of the first local coordinate frame 126, i.e., along its first axis 127, may be mapped to a second transformation of the second three-dimensional digital tooth model 140 in the vestibular direction of the second local coordinate frame 156, i.e., along its first axis 157. In case the local coordinate frame 126, 156 are configured mirror symmetric with respect to a mirror plane, e.g., a sagittal plane, the mapping of the first transformation may correspond to a mirroring at the respective mirror plane. For example, the adjustment mirror plane divides the arrangement of the three-dimensional digital tooth models of the tooth set into two halves.

For mapping the largest shape-deforming adjustment, the local coordinate frames 126, 156 assigned to the three-dimensional digital tooth models 110, 140 may be used as references for determining directions. The largest shape-deforming adjustment may be mapped, e.g., concurrently mapped, to the target three-dimensional digital tooth model, e.g., the first three-dimensional digital tooth models 110, such that a direction of the mapped largest shape-deforming adjustment relative to a target local coordinate frame of the target three-dimensional digital tooth model, e.g., the first local coordinate frame 126 of the first three-dimensional digital tooth models 110, equals a direction of the largest shape-deforming adjustment relative to an origin local coordinate frame of the origin three-dimensional digital tooth model, e.g., the second local coordinate frame 156 of the second three-dimensional digital tooth models 140. For example, a largest shape-deforming adjustment of the second three-dimensional digital tooth model 140 defined along the vestibular direction of the second local coordinate frame 156, i.e., along its first axis 157, may be mapped to a shape-deforming adjustment of the first three-dimensional digital tooth model 110 along the vestibular direction of the first local coordinate frame 126, i.e., along its first axis 127. In case the local coordinate frame 126, 156 are configured mirror symmetric with respect to a mirror plane, e.g., a sagittal plane, the mapping of the largest shape-deforming adjustment may correspond to a mirroring at the respective mirror plane. For example, the mirror plane divides the arrangement of the three-dimensional digital tooth models of the tooth set into two halves.

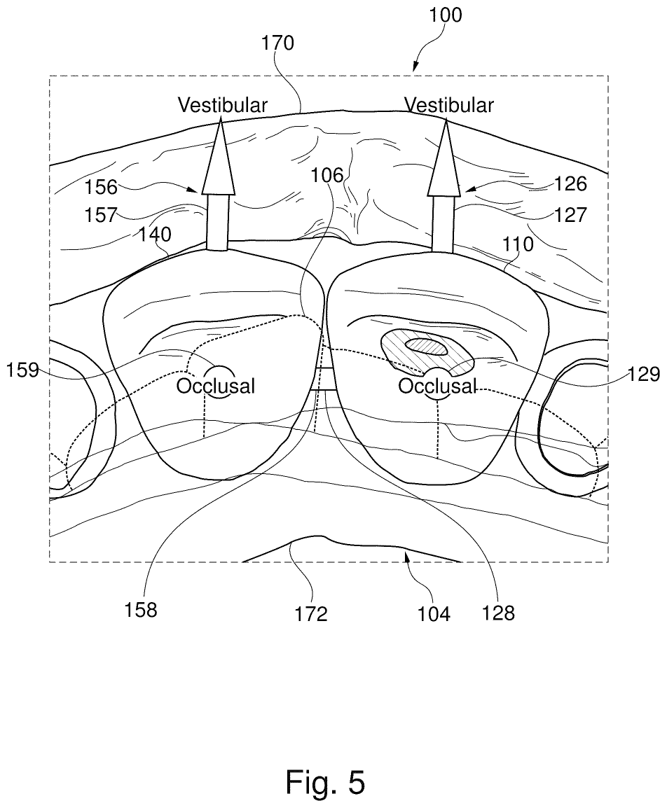

FIG. 5 shows the pair of a first and second three-dimensional digital tooth model 110, 140 of FIG. 4 to which the same largest shape-deforming adjustment has been applied, rather than applying different shape-deforming adjustments as shown in FIG. 4. The same largest shape-deforming adjustment is applied to both three-dimensional digital tooth model 110, 140. In the example, shown in FIG. 5, the largest shape-deforming adjustment is the one required for the second three-dimensional digital tooth model 110, 140. The difference between the two shape-deforming adjustments is illustrated in FIG. 4. Applying the same largest shape-deforming adjustment to both three-dimensional digital tooth models 110, 140, may on the one hand side ensure that none of the three-dimensional digital tooth models 110, 140 has intersections with the antagonistic structure 106 defined by the three-dimensional digital antagonistic model 104 due to the transformations and on the other hand side ensure a symmetrical appearance of the paired three-dimensional digital tooth models 110, 140.

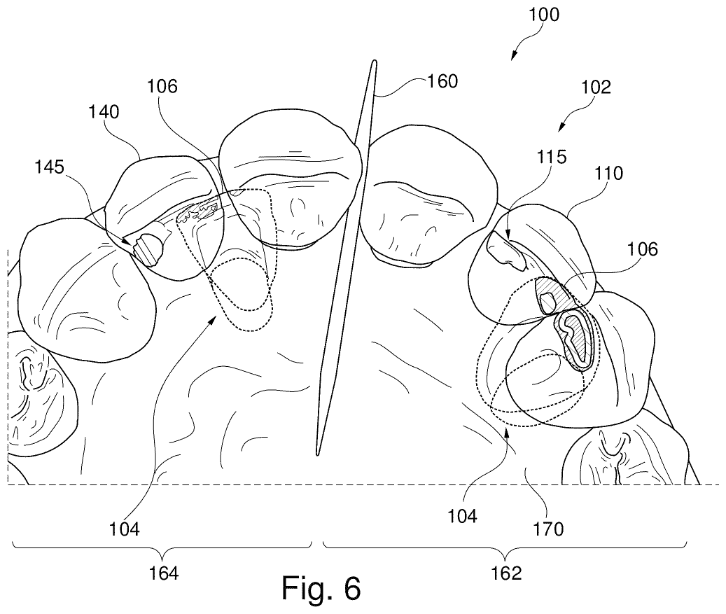

FIG. 6 shows a tooth set 102 with a plurality of three-dimensional digital tooth models, which are configured pair-wise symmetric with respect to a common mirror plane 160. The exemplary mirror plane 160 is a sagittal plane. For example, the mirror plane divides the arrangement of the three-dimensional digital tooth models of the tooth set 102 into two halve 162, 164.

The pair-wise symmetric three-dimensional digital tooth models comprise a first three-dimensional digital tooth model 110, which is paired with a second three-dimensional digital tooth model 140. For example, the local coordinate frames of the three-dimensional digital tooth models may be configured mirror symmetric with respect to the mirror plane 160. The pair-wise symmetric three-dimensional digital tooth models, like first and second three-dimensional digital tooth model 110, 140 may, e.g., be symmetric with respect to their positions, orientations, sizes, and/or shapes relative to the mirror plane 160.

Since the antagonistic structures 106 defined by the three-dimensional digital antagonistic model 104 are not symmetric with respect to the mirror plane 160, the shape-deforming adjustments required for preventing intersections with the three-dimensional digital antagonistic model 104 may differ for the first and second three-dimensional digital tooth model 110, 140.

The first three-dimensional digital tooth model 110, e.g., comprises a shape-deforming adjustment 115 at a first surface section of its oral surface section, which is not required for preventing an intersection of the first three-dimensional digital tooth model 110 with the three-dimensional digital antagonistic model 104. This shape-deforming adjustment 115 rather corresponds to a shape-deforming adjustment required by the second three-dimensional digital tooth model 140 to prevent an intersection with the digital antagonistic model 104 within a first surface section of its oral surface section, which corresponds to the first surface section of the first three-dimensional digital tooth model 110. The first surfaces of the two three-dimensional digital tooth model 110, 140 sections may, e.g., be related by a mirror symmetry with respect to the mirror plane 160. Since within the first surface section intersection of the first three-dimensional digital tooth model 110, no shape-deforming adjustment is required for preventing an intersection of the first three-dimensional digital tooth model 110 with the three-dimensional digital antagonistic model 104, the measure of the shape-deforming adjustment and thus the respective shape-deforming adjustment within the corresponding first surface section of the second three-dimensional digital tooth model 140 is the largest. This largest shape-deforming adjustment is applied to the first three-dimensional digital tooth model 110, while it is also applied to the second three-dimensional digital tooth model 140, for which it was determined. As a result, intersections with the three-dimensional digital antagonistic model 104 within the corresponding first surface sections of the first and second three-dimensional digital tooth model 110, 140 are prevented and the symmetry of the first and second three-dimensional digital tooth model 110, 140 with respect to the mirror plane 160 is maintained.

In addition, the second three-dimensional digital tooth model 140 may, e.g., comprise a shape-deforming adjustment 145 at a second surface section of its oral surface section, which is not required for preventing an intersection of the second three-dimensional digital tooth model 140 with the three-dimensional digital antagonistic model 104. This shape-deforming adjustment 145 rather corresponds to a shape-deforming adjustment required by the first three-dimensional digital tooth model 110 to prevent an intersection with the digital antagonistic model 104 within a second surface section of its oral surface section, which corresponds to the second surface section of the second three-dimensional digital tooth model 140. The second surfaces of the two three-dimensional digital tooth model 110, 140 sections may, e.g., be related by a mirror symmetry with respect to the mirror plane 160. Since within the second surface section intersection of the second three-dimensional digital tooth model 140, no shape-deforming adjustment is required for preventing an intersection of the second three-dimensional digital tooth model 140 with the three-dimensional digital antagonistic model 104, the measure of the shape-deforming adjustment and thus the respective shape-deforming adjustment within the corresponding second surface section of the first three-dimensional digital tooth model 140 is the largest. This largest shape-deforming adjustment is applied to the second three-dimensional digital tooth model 140, while it is also applied to the first three-dimensional digital tooth model 110, for which it was determined. As a result, intersections with the three-dimensional digital antagonistic model 104 within the corresponding second surface sections of the first and second three-dimensional digital tooth model 110, 140 are prevented and the symmetry of the first and second three-dimensional digital tooth model 110, 140 with respect to the mirror plane 160 is maintained.

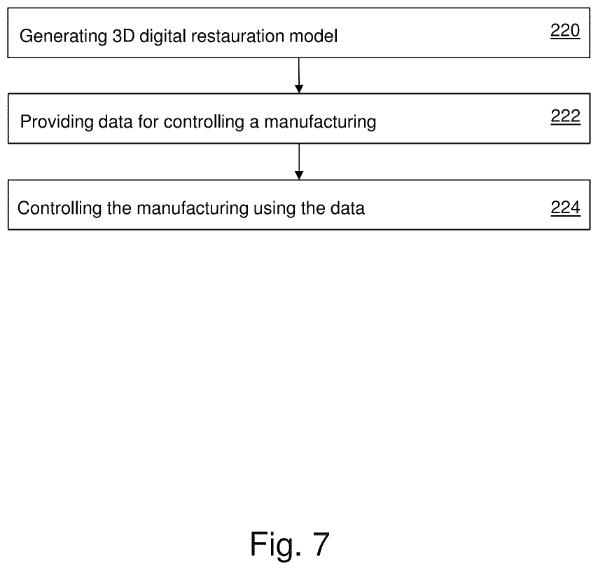

FIG. 7 shows an exemplary method for generating a dental restoration using an adjusted tooth set of three-dimensional digital tooth models for the dental restoration. The adjusted tooth set of three-dimensional digital tooth models comprises an adjusted first and second three-dimensional digital tooth models, which have been adjusted using, e.g., the method of FIG. 1. In block 220, a three-dimensional digital restoration model of the dental restoration comprising the tooth set with the adjusted first and second three-dimensional digital tooth models is generated. In block 222, data for controlling the manufacturing of a physical dental restoration is provided. The data defines the three-dimensional digital restoration model as a template for the physical dental restoration. For example, the data comprises the three-dimensional digital restoration model as the template for the physical dental restoration.

In block 224, the physical dental restoration is manufactured using the data provided for controlling the manufacturing. The manufactured physical dental restoration is a physical copy of the template defined by the provided data. For the manufacturing of the dental restoration, e.g., a computer-controlled additive and/or subtractive method may be used. For example, the dental restoration and/or elements of the dental restoration may be manufactured using one of the following: machining, 3D printing, casting. For the manufacturing, a manufacturing system may be used, which may comprise one or more manufacturing devices. The one or more manufacturing devices of the manufacturing system may comprise one or more of the following: a machining device, a 3D printing device.

For example, the dental restoration is a set of crowns, in particular a set of implant-based crowns. For example, the dental restoration is a bridge. For example, the bridge is a partial bridge comprising a part of a dental arch. For example, the bridge is a complete bridge comprising a complete dental arch.

For example, the dental restoration is a denture. For example, the denture is a partial denture comprising a part of a dental arch. For example, the denture is a full denture comprising a complete dental arch.

For example, the denture is a removable denture. For example, the denture is a removable partial denture or a removable complete denture. For example, the denture is a fixed denture, i.e., implant-based denture. For example, the denture is a fixed partial denture or a fixed complete denture.

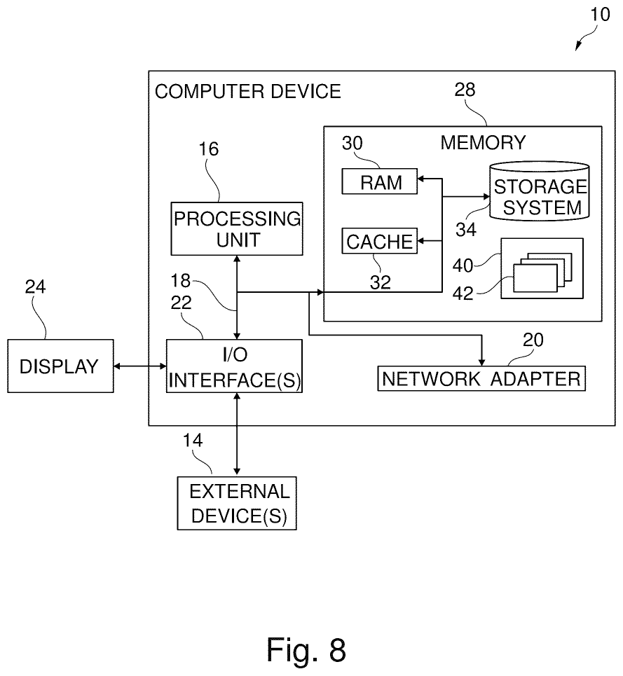

FIG. 8 shows a schematic diagram of an exemplary computer device 10 for adjusting two or more three-dimensional digital tooth models of a tooth set of three-dimensional digital tooth models of a jaw. The tooth set may be a tooth set for a dental restoration. The computer device 10 may be operational with numerous other general-purpose or special-purpose computing system environments or configurations. Computer device 10 may be described in the general context of computer device executable instructions, such as program modules comprising executable program instructions, being executable by the computer device 10. Generally, program modules may include routines, programs, objects, components, logic, data structures, and so on that perform particular tasks or implement particular abstract data types. Computer device 10 may be practiced in distributed computing environments where tasks are performed by remote processing devices that are linked through a communications network. In a distributed computing environment, program modules may be located in both local and remote computer device storage media including memory storage devices.

In FIG. 8, computer device 10 is shown in the form of a general-purpose computing device. The components of computer device 10 may include, but are not limited to, one or more processors or processing units 16, a system memory 28, and a bus 18 that couples various system components including system memory 28 to processing unit 16. Bus 18 represents one or more of any of several types of bus structures, including a memory bus or memory controller, a peripheral bus, an accelerated graphics port, and a processing unit or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer device 10 may comprise a variety of computer device readable storage media. Such media may be any available storage media accessible by computer device 10, and include both volatile and non-volatile storage media, removable and non-removable storage media.

A system memory 28 may include computer device readable storage media in the form of volatile memory, such as random-access memory (RAM) 30 and/or cache memory 32. Computer device 10 may further include other removable/non-removable, volatile/non-volatile computer device storage media. For example, storage system 34 may be provided for reading from and writing to a non-removable, non-volatile magnetic media also referred to as a hard drive. For example, a magnetic disk drive for reading from and writing to a removable, non-volatile magnetic disk, e.g., a floppy disk, and an optical disk drive for reading from or writing to a removable, non-volatile optical disk such as a CD-ROM, DVD-ROM or other optical storage media may be provided. In such instances, each storage medium may be connected to bus 18 by one or more data media interfaces. Memory 28 may, e.g., include a three-dimensional digital dentition model comprising the tooth set and defining an arrangement of the three-dimensional digital tooth models of the tooth set. The three-dimensional digital dentition model may comprise a three-dimensional digital antagonistic model of one or more antagonistic structures of an antagonistic jaw and define an arrangement of the three-dimensional digital antagonistic model relative to the arrangement of the three-dimensional digital tooth models of the tooth set The three-dimensional digital dentition model may e.g., be a tissue model of the defining the intraoral tissue of the patient, on which the dental restoration defined by the three-dimensional digital denture model is to be arranged. This tissue model may be supplemented by three-dimensional digital tooth models of the tooth set for the dental restoration.

Program 40 may have a set of one or more program modules 42 and by way of example be stored in memory 28. The program modules 42 may comprise an operating system, one or more application programs, other program modules, and/or program data. Each of these program modules 42, i.e., the operating system, the one or more application programs, the other program modules, and/or the program data or some combination thereof, may include an implementation of a networking environment. One or more of the program modules 42 may be configured for adjusting two or more three-dimensional digital tooth models of a tooth set of three-dimensional digital tooth models of a jaw. The program modules 42 may, e.g., be configured to control the computer device 10 to receive a three-dimensional digital dentition model comprising the tooth set and defining an arrangement of the three-dimensional digital tooth models of the tooth set. The three-dimensional digital dentition model further comprises a three-dimensional digital antagonistic model of one or more antagonistic structures of an antagonistic jaw and defines an arrangement of the three-dimensional digital antagonistic model relative to the arrangement of the three-dimensional digital tooth models of the tooth set. A first three-dimensional digital tooth model of the tooth set descriptive of a first tooth is paired with a second three-dimensional digital tooth model of the tooth set descriptive of a second tooth being a contralateral counterpart tooth of the first tooth.

An input defining a first transformation to be applied to the first three-dimensional digital tooth model is received. For the first transformation a first measure of an additional first shape-deforming adjustment of a shape of the first three-dimensional digital tooth model is determined, which is required for preventing an intersection of the first three-dimensional digital tooth model with the three-dimensional digital antagonistic model due to the first transformation. Using the input, a second transformation to be applied to the second three-dimensional digital tooth model is determined. The determining of the second transformation comprises a mapping of the first transformation to the second three-dimensional digital tooth model. For the second transformation a second measure of an additional second shape-deforming adjustment of a shape of the second three-dimensional digital tooth model is determined, which is required for preventing an intersection of the second three-dimensional digital tooth model with the three-dimensional digital antagonistic model due to the second transformation.

A largest one of the two shape-deforming adjustments is determined, which comprises a largest one of the two measures. The first three-dimensional digital tooth model and the second three-dimensional digital tooth model are adjusted. For example, the first three-dimensional digital tooth model and the second three-dimensional digital tooth model may be adjusted concurrently, i.e., simultaneously. The adjusting comprises applying in combination the first transformation and the largest shape-deforming adjustment to the first three-dimensional digital tooth model. The first transformation and the largest shape-deforming adjustment are, e.g., applied concurrently in combination to the first three-dimensional digital tooth model. Furthermore, in combination the second transformation and the same largest shape-deforming adjustment are applied to the second three-dimensional digital tooth model. The second transformation and the same largest shape-deforming adjustment are, e.g., applied concurrently in combination to the second three-dimensional digital tooth model.

One of the program modules 42 may, e.g., further be configured for generating a three-dimensional digital dental restoration model of the dental restoration to be manufactured using the adjusted three-dimensional digital denture model provided for manufacturing the dental restoration. One of the program modules 42 may, e.g., be configured to train a machine learning module to be trained. The machine learning module to be trained may, e.g., be an untrained machine learning module, a pre-trained machine learning module or a partially trained machine learning module.

Computer device 10 may further communicate with one or more external devices 14 such as a keyboard, a pointing device, like a mouse, and a display 24 enabling a user to interact with computer device 10. Such communication can occur via input/output (I/O) interfaces 22. Computer device 10 may further communicate with one or more networks such as a local area network (LAN), a general wide area network (WAN), and/or a public network, like the Internet, via network adapter 20. Network adapter 20 may communicate with other components of computer device 10 via bus 18. It should be understood that although not shown, other hardware and/or software components could be used in conjunction with computer device 10. For example, computer device 10 may provide one or more computing functions as cloud server. For example, the computer device 10 may receive from a requesting computer device the input for executing a cloud-based computation. For example, computer device 10 may be configured for intensive computations. The result of the cloud-based computation may be transmitted back to the requesting computer device, e.g., via a video stream. For example, computer device 10 may request as a client one or more computing functions to be executed by a cloud server. For example, the computer device 10 may send as a requesting computer device the input to a cloud server for executing a cloud-based computation. For example, the cloud server may be configured for intensive computations. The result of the cloud-based computation may be received by computer device 10, e.g., via a video stream.

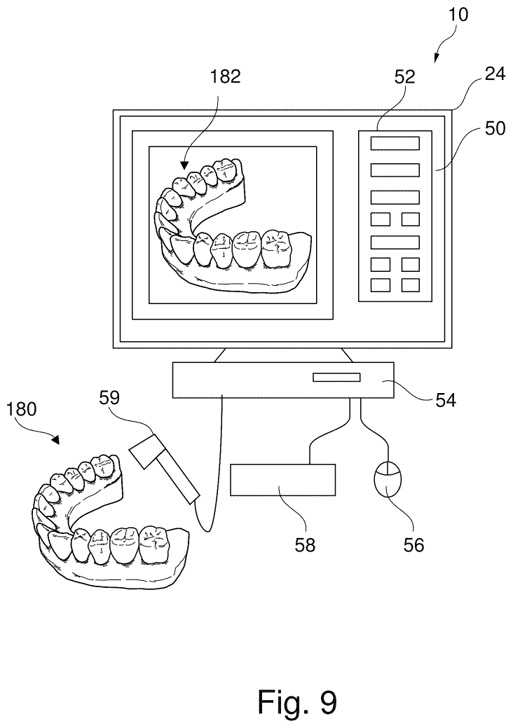

FIG. 9 shows an exemplary computer device 10 for adjusting two or more three-dimensional digital tooth models of a tooth set of three-dimensional digital tooth models of a jaw. The computer device 10 may, e.g., be configured as shown in FIG. 8. The computer device 10 may comprise a hardware component 54 comprising one or more processing units as well as a memory storing machine-executable program instructions. Execution of the program instructions by the one or more processing units may cause the one or more processing units to control the computer device 10 to adjust the two or more three-dimensional digital tooth models of the tooth set of three-dimensional digital tooth models of the jaw. For this purpose, a three-dimensional digital dentition model comprising the tooth set and defining an arrangement of the three-dimensional digital tooth models of the tooth set is received. The three-dimensional digital dentition model further comprises a three-dimensional digital antagonistic model of one or more antagonistic structures of an antagonistic jaw and defines an arrangement of the three-dimensional digital antagonistic model relative to the arrangement of the three-dimensional digital tooth models of the tooth set. A first three-dimensional digital tooth model of the tooth set descriptive of a first tooth is paired with a second three-dimensional digital tooth model of the tooth set descriptive of a second tooth being a contralateral counterpart tooth of the first tooth. An input defining a first transformation to be applied to the first three-dimensional digital tooth model is received. For the first transformation a first measure of an additional first shape-deforming adjustment of a shape of the first three-dimensional digital tooth model is determined, which is required for preventing an intersection of the first three-dimensional digital tooth model with the three-dimensional digital antagonistic model due to the first transformation. Using the input, a second transformation to be applied to the second three-dimensional digital tooth model is determined. The determining of the second transformation comprises a mapping of the first transformation to the second three-dimensional digital tooth model. For the second transformation a second measure of an additional second shape-deforming adjustment of a shape of the second three-dimensional digital tooth model is determined, which is required for preventing an intersection of the second three-dimensional digital tooth model with the three-dimensional digital antagonistic model due to the second transformation.

A largest one of the two shape-deforming adjustments is determined, which comprises a largest one of the two measures. The first three-dimensional digital tooth model and the second three-dimensional digital tooth model are adjusted. For example, the first three-dimensional digital tooth model and the second three-dimensional digital tooth model may be adjusted concurrently, i.e., simultaneously. The adjusting comprises applying in combination the first transformation and the largest shape-deforming adjustment to the first three-dimensional digital tooth model. The first transformation and the largest shape-deforming adjustment are, e.g., applied concurrently in combination to the first three-dimensional digital tooth model. Furthermore, in combination the second transformation and the same largest shape-deforming adjustment are applied to the second three-dimensional digital tooth model. The second transformation and the same largest shape-deforming adjustment are, e.g., applied concurrently in combination to the second three-dimensional digital tooth model.

The computer device 10 may further comprise one or more input devices, like a keyboard 58 and a mouse 56, enabling a user to interact with the computer device 10. The input devices may, e.g., be configured for receiving the input defining a first transformation to be applied to the first three-dimensional digital tooth model.

Furthermore, the computer device 10 may comprise one or more output devices, like a display 24 providing a graphical user interface 50 with control elements 52, e.g., GUI elements, enabling the user to control the adjusting of the arrangement of a plurality of three-dimensional digital tooth models for the dental restoration. For example, the three-dimensional digital dentition model comprising the tooth set may be displayed on display 24. This three-dimensional digital dentition model may, e.g., comprise a three-dimensional digital antagonistic model of one or more antagonistic structures of an antagonistic jaw model and define an arrangement of the three-dimensional digital antagonistic model relative to the arrangement of the three-dimensional digital tooth models of the tooth set. The three-dimensional digital dentition model may, e.g., comprise a three-dimensional digital tissue model, on which the tooth set is arranged. In addition, the resulting adjusted three-dimensional digital dentition model with the adjusted first and second three-dimensional digital tooth model may, e.g., be displayed on display 24.

The computer device 10 may further comprise an exemplary scanner 59 configured for scanning a patient's mouth and/or imprints of the patient's intraoral tissue. The scanner 59 may, e.g., comprise an optical scanner configured for scanning, e.g., a patient's oral cavity, an imprint of a patient's oral cavity and/or a positive of a patient's oral cavity generated using an imprint. For example, the scanner may be configured for scanning intraoral tissue 180 within a patient's oral cavity comprising intraoral tissue. The intraoral tissue being scanned may, e.g., comprise hard and/or soft tissue. Hard tissue may, e.g., comprise teeth, while soft tissue may, e.g., comprise gingiva tissue. The intraoral tissue being scanned may, e.g., comprise one or more jaws of the patient, i.e., a mandible and/or a maxilla. A jaw being scanned may, e.g., be an edentulous jaw or a jaw comprising one or more teeth. The jaw being scanned may, e.g., comprise a full dental arch. Alternatively, scanner 59 may be configured for scanning an imprint of the intraoral tissue and/or a positive of the intraoral tissue generated using an imprint. The scan data acquired using the scanner 59 may, e.g., be used for generating the three-dimensional digital tissue model 182. This three-dimensional digital tissue model 182 may be used for providing the three-dimensional digital dentition model 100. Providing the three-dimensional digital dentition model 100 may, e.g., comprise adding the tooth set for the dental restoration to the three-dimensional digital tissue model 182. The three-dimensional digital tooth models comprised by the tooth set may, e.g., be provided using three-dimensional digital tooth models in form of library teeth provided by a tooth library. The three-dimensional digital tooth models comprised by the tooth set may, e.g., be provided using scans of natural teeth or of physical tooth models. The three-dimensional digital tooth models comprised by the tooth set may, e.g., be generated from scratch.

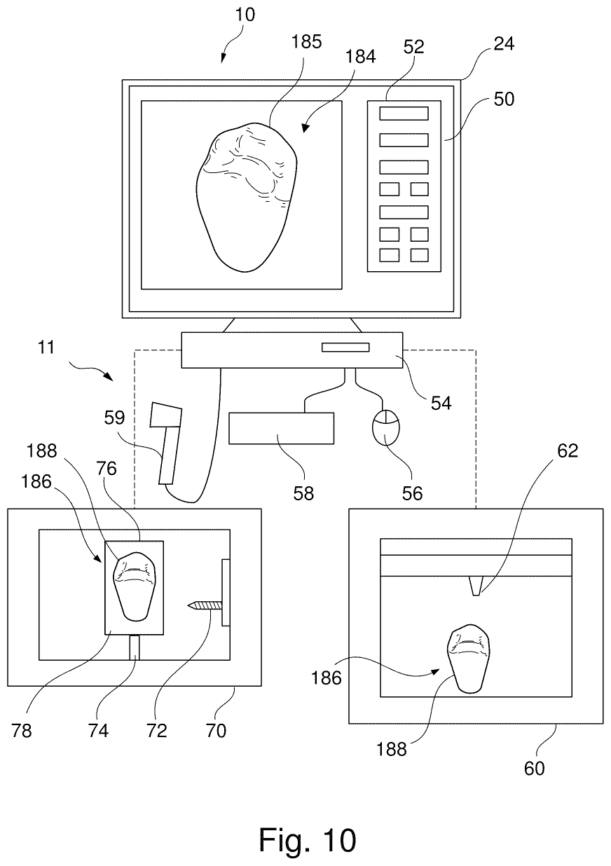

FIG. 10 shows an exemplary manufacturing system 11 for manufacturing a dental restoration 186 or at least one or more elements 188 of a dental restoration 186. A three-dimensional digital dental restoration model 184 comprising one or more three-dimensional digital restoration elements 185 defining one or more elements 188 of the dental restoration 186, like e.g., a physical artificial tooth, may be provided. This three-dimensional digital dental restoration model 184 may, e.g., be used as a template for manufacturing the dental restoration 186 and/or one or more dental restoration elements 188, e.g., one or more physical artificial teeth, as a physical copy of the template. For generating the dental restoration model 184 comprising the one or more dental restoration elements 185, e.g., the adjusted three-dimensional digital dentition model may be used.

The manufacturing system 11 may comprise the computer device 10 of FIG. 8 and/or FIG. 9. The computer device 10 may further be configured to control one or more manufacturing devices 60, 70. For example, the manufacturing system 11 may comprise a manufacturing device in form of a machining device 70 controlled by the computer device 10. The machining device 70 may be configured to machining a blank 76 using one or more machining tools 72. The blank 76 of raw material 78, may be provided using a holding device 74 and cut into a desired shape and size of the element to be manufactured, e.g., a physical artificial tooth, as an element 188 of the dental restoration 186 as defined by the three-dimensional digital dental restoration model 184. The machining tool 72 may, e.g., be a milling tool.

For example, the manufacturing system 11 may comprise a manufacturing device in form of a 3D printing device 60. The 3D printing device 60 may be controlled by the computer device 10 and configured to print an element to be manufactured, e.g., a physical artificial tooth, as an element 188 of the dental restoration 186 as defined by the three-dimensional digital dental restoration model 184. The 3D printing device 60 may comprise a printing element 62 configured to print the respective element 188, like a physical artificial tooth, layer by layer. The printing element 62 may, e.g., comprise a nozzle configured for distributing printing material.

In case the element to be manufactured using the 3D printing device 60 is made using metal, the 3D printing device 60 may, e.g., be configured for executing selective laser sintering or melting. Selective laser sintering uses a laser for sintering a powdered material, aiming the laser automatically at points in space defined by a three-dimensional digital model of the element to be printed. The laser energy may result in a local sintering or melting of the powdered material, binding the material together to create a solid structure. For example, the printing element 62 of the 3D printing device 60 may comprise a laser and/or a distributing device for distributing the powdered material.

For example, the three-dimensional digital dental restoration model 184 may be used as a positive to define a negative of the physical dental restoration 186 and/or of one or more dental restoration elements 188, e.g., one or more physical artificial teeth, in form of a negative three-dimensional digital dental restoration model and/or of one or more negative three-dimensional digital dental restoration element models, respectively. The negative three-dimensional digital dental restoration model and/or one or more negative three-dimensional digital dental restoration element models may be used to manufacture, e.g., using machining device 70 or 3D printing device 60, one or more casting matrices. The one or more casting matrices may, e.g., be configured for casting the dental restoration 186 and/or one or more dental restoration elements 188, like a physical artificial tooth, by inserting restoration material into the casting matrix and curing the inserted restoration material.

While the invention has been illustrated and described in detail in the drawings and foregoing description, such illustration and description are to be considered illustrative or exemplary and not restrictive; the invention is not limited to the disclosed embodiments.

While the invention has been illustrated and described in detail in the drawings and foregoing description, such illustration and description are to be considered illustrative or exemplary and not restrictive; the invention is not limited to the disclosed examples.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

A single processor or other unit may fulfill the functions of several items recited in the claims. A computer program may be stored/distributed on a suitable medium, such as an optical storage medium or a solid-state medium supplied together with or as part of other hardware, but may also be distributed in other forms, such as via the Internet or other wired or wireless telecommunication systems.

As will be appreciated by one skilled in the art, aspects of the present invention may be embodied as an apparatus, method, computer program or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer executable code embodied thereon. A computer program comprises the computer executable code or “program instructions”.

Any combination of one or more computer readable medium(s) may be utilized. The computer readable medium may be a computer readable signal medium or a computer readable storage medium. A “computer-readable storage medium” as used herein encompasses any tangible storage medium which may store instructions which are executable by a processor of a computing device. The computer-readable storage medium may be referred to as a computer-readable non-transitory storage medium. The computer-readable storage medium may also be referred to as a tangible computer readable medium. For example, a computer-readable storage medium may also be able to store data which is able to be accessed by the processor of the computing device. Examples of computer-readable storage media include, but are not limited to: a floppy disk, a magnetic hard disk drive, a solid-state hard disk, flash memory, a USB thumb drive, Random Access Memory (RAM), Read Only Memory (ROM), an optical disk, a magneto-optical disk, and the register file of the processor. Examples of optical disks include Compact Disks (CD) and Digital Versatile Disks (DVD), for example CD-ROM, CD-RW, CD-R, DVD-ROM, DVD-RW, or DVD-R disks. A further example of an optical disk may be a Blu-ray disk. The term computer readable-storage medium also refers to various types of recording media capable of being accessed by the computer device via a network or communication link. For example, a data may be retrieved over a modem, over the internet, or over a local area network. Computer executable code embodied on a computer readable medium may be transmitted using any appropriate medium, including but not limited to wireless, wireline, optical fiber cable, RF, etc., or any suitable combination of the foregoing.

A computer readable signal medium may include a propagated data signal with computer executable code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

“Computer memory” or “memory” is an example of a computer-readable storage medium. Computer memory is any memory which is directly accessible to a processor. “Computer storage” or “storage” is a further example of a computer-readable storage medium. Computer storage is any non-volatile computer-readable storage medium. For example, computer storage may also be computer memory or vice versa.

A “processor” as used herein encompasses an electronic component which is able to execute a program or machine executable instruction or computer executable code. References to the computing device comprising “a processor” should be interpreted as possibly containing more than one processor or processing core. The processor may for instance be a multi-core processor. A processor may also refer to a collection of processors within a single computer device or distributed amongst multiple computer devices. The term computing device should also be interpreted to possibly refer to a collection or network of computing devices each comprising a processor or processors. The computer executable code may be executed by multiple processors that may be within the same computing device or which may even be distributed across multiple computing devices.

Computer executable code may comprise machine executable instructions or a program which causes a processor to perform an aspect of the present invention. Computer executable code for carrying out operations for aspects of the present invention may be written in any combination of one or more programming languages, including an object-oriented programming language such as Java, Smalltalk, C++ or the like and conventional procedural programming languages, such as the “C” programming language or similar programming languages and compiled into machine executable instructions. In some instances, the computer executable code may be in the form of a high-level language or in a pre-compiled form and be used in conjunction with an interpreter which generates the machine executable instructions on the fly.

The computer executable code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Generally, the program instructions can be executed on one processor or on several processors. In the case of multiple processors, they can be distributed over several different entities like clients, servers etc. Each processor could execute a portion of the instructions intended for that entity. Thus, when referring to a system or process involving multiple entities, the computer program or program instructions are understood to be adapted to be executed by a processor associated or related to the respective entity.

A “user interface” as used herein is an interface which allows a user or operator to interact with a computer or computer device. A ‘user interface’ may also be referred to as a ‘human interface device.’ A user interface may provide information or data to the operator and/or receive information or data from the operator. A user interface may enable input from an operator to be received by the computer and may provide output to the user from the computer. In other words, the user interface may allow an operator to control or manipulate a computer and the interface may allow the computer to indicate the effects of the operator's control or manipulation. The display of data or information on a display or a graphical user interface is an example of providing information to an operator. The receiving of data through a keyboard, mouse, trackball, touchpad, pointing stick, graphics tablet, joystick, gamepad, webcam, headset, gear sticks, steering wheel, pedals, wired glove, dance pad, remote control, one or more switches, one or more buttons, and accelerometer are all examples of user interface components which enable the receiving of information or data from an operator.

A GUI element is a data object some of which's attributes specify the shape, layout and/or behavior of an area displayed on a graphical user interface, e.g., a screen. A GUI element can be a standard GUI element such as a button, a text box, a tab, an icon, a text field, a pane, a check-box item or item group or the like. A GUI element can likewise be an image, an alphanumeric character or any combination thereof. At least some of the properties of the displayed GUI elements depend on the data value aggregated on the group of data object said GUI element represents.

Aspects of the present invention are described with reference to flowchart illustrations and/or block diagrams of methods, apparatus (systems) and computer program products. It will be understood that each block or a portion of the blocks of the flowchart, illustrations, and/or block diagrams, can be implemented by computer program instructions in form of computer executable code when applicable. It is further understood that, when not mutually exclusive, combinations of blocks in different flowcharts, illustrations, and/or block diagrams may be combined. These computer program instructions may be provided to a processor of a general-purpose computer, special-purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer-implemented process such that the instructions which execute on the computer or other programmable apparatus provide processes for implementing the functions/acts specified in the flowchart and/or block diagram block or blocks.

Although the invention may have been described in reference to specific examples, it should be understood that the invention is not limited to these examples only and that many variations of these examples may be readily envisioned by the skilled person after having read the present disclosure. The invention may thus further be described without limitation and by way of example only by the following embodiments. The following exemplary embodiments may contain preferred embodiments. Accordingly, the term “feature combination” as used therein may refer to such a “preferred embodiment”.

1. A computer-implemented method for adjusting two or more three-dimensional digital tooth models of a tooth set of three-dimensional digital tooth models of a jaw, the tooth set being a tooth set for a dental restoration, the method comprising:
receiving a three-dimensional digital dentition model comprising the tooth set and defining an arrangement of the three-dimensional digital tooth models of the tooth set, the three-dimensional digital dentition model further comprising a three-dimensional digital antagonistic model of one or more antagonistic structures of an antagonistic jaw and defining an arrangement of the three-dimensional digital antagonistic model relative to the arrangement of the three-dimensional digital tooth models of the tooth set,
pairing a first three-dimensional digital tooth model of the tooth set descriptive of a first tooth with a second three-dimensional digital tooth model of the tooth set descriptive of a second tooth being a contralateral counterpart tooth of the first tooth,
receiving an input defining a first transformation to be applied to the first three-dimensional digital tooth model,
determining for the first transformation a first measure of an additional first shape-deforming adjustment of a shape of the first three-dimensional digital tooth model required for preventing an intersection of the first three-dimensional digital tooth model with the three-dimensional digital antagonistic model due to the first transformation,
determining, using the input, a second transformation to be applied to the second three-dimensional digital tooth model, the determining of the second transformation comprising a mapping of the first transformation to the second three-dimensional digital tooth model,
determining for the second transformation a second measure of an additional second shape-deforming adjustment of a shape of the second three-dimensional digital tooth model required for preventing an intersection of the second three-dimensional digital tooth model with the three-dimensional digital antagonistic model due to the second transformation,
determining a largest one of the two shape-deforming adjustments comprising a largest one of the two measures,
adjusting the first and second three-dimensional digital tooth model, the adjusting comprising:
   applying in combination the first transformation and the largest shape-deforming adjustment to the first three-dimensional digital tooth model; and
   applying in combination the second transformation and the same largest shape-deforming adjustment to the second three-dimensional digital tooth model.

2. The method of feature combination 1, the applying of the largest shape-deforming adjustment comprising a mapping of the largest shape-deforming adjustment from an origin three-dimensional digital tooth model to a target three-dimensional digital tooth model, the origin three-dimensional digital tooth model being the one of the first or second three-dimensional digital tooth model, for which the largest shape-deforming adjustment is determined, and the target three-dimensional digital tooth model being the other one of the first or second three-dimensional digital tooth model.

3. The method of feature combination 2, the mapping of the largest shape-deforming adjustment comprising a mirroring of the largest shape-deforming adjustment onto the target three-dimensional digital tooth model at an adjustment mirror plane arranged between the origin and the target three-dimensional digital tooth model.

4. The method of any of feature combinations 2 to 3, the largest shape-deforming adjustment being mapped to the target three-dimensional digital tooth model, such that a direction of the mapped largest shape-deforming adjustment relative to a target local coordinate frame of the target three-dimensional digital tooth model equals a direction of the largest shape-deforming adjustment relative to an origin local coordinate frame of the origin three-dimensional digital tooth model.

5. The method of any of the previous feature combinations, the first measure being a first distance of a displacing of a first surface section of the first three-dimensional digital tooth model along a first reference direction, which results from the first shape-deforming adjustment, the second measure being a second distance of a displacing of a second surface section of the second three-dimensional digital tooth model along a second reference direction, which results from the second shape-deforming adjustment.

6. The method of feature combination 5, the first reference direction and the second reference direction being related by a mirror symmetry defined by the adjustment mirror plane.

7. The method of any of feature combinations 5 to 6, a definition of the second reference direction relative to a second local coordinate frame of the second three-dimensional digital tooth model equaling a definition of the first reference direction relative to a first local coordinate frame of the first three-dimensional digital tooth model.

8. The method of any of feature combinations 5 to 7, the first reference direction being a vestibular direction of the first three-dimensional digital tooth model and the second reference direction being a vestibular direction of the second three-dimensional digital tooth model.

9. The method of any of feature combinations 5 to 8, the displacing of the first surface section of the first three-dimensional digital tooth model comprising a displacing of a first vertex of a first mesh defining the first surface section, the displacing of the second surface section of the second three-dimensional digital tooth model comprising a displacing of a second vertex of a second mesh defining the second surface section.

10. The method of feature combination 9, the first measure being the first distance of the displacing of the first vertex, the second measure being the second distance of the displacing of the second vertex.

11. The method of any of feature combinations 9 to 10, the first and second vertex being corresponding vertices, the method further comprising determining the corresponding first and second vertices, the determining of the corresponding first and second vertices comprising using one of the following: a ray intersection, a closest point determination, an interpolation, a three-dimensional coordinate transformation.

12. The method of any of feature combinations 9 to 11, the displacing of the first surface section comprising a displacing of a first plurality of first vertices of the first mesh comprising the first vertex, the displacing of the second surface section comprising a displacing of a second plurality of second vertices of the second mesh comprising the second vertex,
the determining of the first measure being executed vertex-wise per displaced first vertex of the first plurality of first vertices, the determining of the second measure being executed vertex-wise per displaced second vertex of the second plurality of second vertices,
the determining of the largest shape-deforming adjustment being executed vertex-wise and comprising a comparison of the vertex-wise determined first measure with the corresponding vertex-wise determined second measure,
the applying of the largest shape-deforming adjustment being executed vertex-wise.

13. The method of any of the previous feature combinations, the first transformation comprising one or more of the following: a translation, a rotation, a scaling, a deforming, an adding of tooth material, a removing of tooth material, a modification of a surface structure.

14. The method of any of the previous feature combinations, the mapping of the first transformation comprising a mirroring of the first transformation to the second three-dimensional digital tooth model at a transformation mirror plane arranged between the first and second three-dimensional digital tooth model.

15. The method of any of the previous feature combinations, the three-dimensional digital antagonistic model comprising an antagonistic tooth set of three-dimensional digital antagonistic tooth models of the antagonistic jaw arranged in an antagonistic arrangement.

16. The method of any of the previous feature combinations, the method further comprising:
generating a three-dimensional digital restoration model of the dental restoration comprising the tooth set with the adjusted first and second three-dimensional digital tooth models,
providing data for controlling a manufacturing of a physical dental restoration, the data defining the three-dimensional digital restoration model as a template for the physical dental restoration.

17. The method of feature combination 16, the method further comprising a manufacturing of the physical dental restoration using the data provided for controlling the manufacturing with the manufactured physical dental restoration being a physical copy of the template defined by the provided data.

18. A computer program product for adjusting two or more three-dimensional digital tooth models of a tooth set of three-dimensional digital tooth models of a jaw, the tooth set being a tooth set for a dental restoration,
the computer program product comprising a non-transitory computer readable storage medium having program instructions embodied therewith, the program instructions being executable by a processing unit of a computer device to cause the computer device to:
receive a three-dimensional digital dentition model comprising the tooth set and defining an arrangement of the three-dimensional digital tooth models of the tooth set, the three-dimensional digital dentition model further comprising a three-dimensional digital antagonistic model of one or more antagonistic structures of an antagonistic jaw and defining an arrangement of the three-dimensional digital antagonistic model relative to the arrangement of the three-dimensional digital tooth models of the tooth set,
pair a first three-dimensional digital tooth model of the tooth set descriptive of a first tooth with a second three-dimensional digital tooth model of the tooth set descriptive of a second tooth being a contralateral counterpart tooth of the first tooth,
receive an input defining a first transformation to be applied to the first three-dimensional digital tooth model,
determine for the first transformation a first measure of an additional first shape-deforming adjustment of a shape of the first three-dimensional digital tooth model required for preventing an intersection of the first three-dimensional digital tooth model with the three-dimensional digital antagonistic model due to the first transformation,
determine, using the input, a second transformation to be applied to the second three-dimensional digital tooth model, the determining of the second transformation comprising a mapping of the first transformation to the second three-dimensional digital tooth model,
determine for the second transformation a second measure of an additional second shape-deforming adjustment of a shape of the second three-dimensional digital tooth model required for preventing an intersection of the second three-dimensional digital tooth model with the three-dimensional digital antagonistic model due to the second transformation,
determine a largest one of the two shape-deforming adjustments comprising a largest one of the two measures,
adjust the first and second three-dimensional digital tooth model, the adjusting comprising:
   applying in combination the first transformation and the largest shape-deforming adjustment to the first three-dimensional digital tooth model; and
   applying in combination the second transformation and the same largest shape-deforming adjustment to the second three-dimensional digital tooth model.

19. A computer program for adjusting two or more three-dimensional digital tooth models of a tooth set of three-dimensional digital tooth models of a jaw, the tooth set being a tooth set for a dental restoration,
the computer program comprising program instructions executable by a processing unit of a computer device to cause the computer device to:
receive a three-dimensional digital dentition model comprising the tooth set and defining an arrangement of the three-dimensional digital tooth models of the tooth set, the three-dimensional digital dentition model further comprising a three-dimensional digital antagonistic model of one or more antagonistic structures of an antagonistic jaw and defining an arrangement of the three-dimensional digital antagonistic model relative to the arrangement of the three-dimensional digital tooth models of the tooth set,
pair a first three-dimensional digital tooth model of the tooth set descriptive of a first tooth with a second three-dimensional digital tooth model of the tooth set descriptive of a second tooth being a contralateral counterpart tooth of the first tooth,
receive an input defining a first transformation to be applied to the first three-dimensional digital tooth model,
determine for the first transformation a first measure of an additional first shape-deforming adjustment of a shape of the first three-dimensional digital tooth model required for preventing an intersection of the first three-dimensional digital tooth model with the three-dimensional digital antagonistic model due to the first transformation,
determine, using the input, a second transformation to be applied to the second three-dimensional digital tooth model, the determining of the second transformation comprising a mapping of the first transformation to the second three-dimensional digital tooth model,
determine for the second transformation a second measure of an additional second shape-deforming adjustment of a shape of the second three-dimensional digital tooth model required for preventing an intersection of the second three-dimensional digital tooth model with the three-dimensional digital antagonistic model due to the second transformation,
determine a largest one of the two shape-deforming adjustments comprising a largest one of the two measures,
adjust the first and second three-dimensional digital tooth model, the adjusting comprising:
   applying in combination the first transformation and the largest shape-deforming adjustment to the first three-dimensional digital tooth model; and
   applying in combination the second transformation and the same largest shape-deforming adjustment to the second three-dimensional digital tooth model.

20. A computer device for adjusting two or more three-dimensional digital tooth models of a tooth set of three-dimensional digital tooth models of a jaw, the tooth set being a tooth set for a dental restoration,
the computer device comprising a processing unit and a memory storing program instructions executable by the processing unit, execution of the program instructions by the processing unit causing the computer device to:
receive a three-dimensional digital dentition model comprising the tooth set and defining an arrangement of the three-dimensional digital tooth models of the tooth set, the three-dimensional digital dentition model further comprising a three-dimensional digital antagonistic model of one or more antagonistic structures of an antagonistic jaw and defining an arrangement of the three-dimensional digital antagonistic model relative to the arrangement of the three-dimensional digital tooth models of the tooth set,
pair a first three-dimensional digital tooth model of the tooth set descriptive of a first tooth with a second three-dimensional digital tooth model of the tooth set descriptive of a second tooth being a contralateral counterpart tooth of the first tooth,
receive an input defining a first transformation to be applied to the first three-dimensional digital tooth model,
determine for the first transformation a first measure of an additional first shape-deforming adjustment of a shape of the first three-dimensional digital tooth model required for preventing an intersection of the first three-dimensional digital tooth model with the three-dimensional digital antagonistic model due to the first transformation,
determine, using the input, a second transformation to be applied to the second three-dimensional digital tooth model, the determining of the second transformation comprising a mapping of the first transformation to the second three-dimensional digital tooth model,
determine for the second transformation a second measure of an additional second shape-deforming adjustment of a shape of the second three-dimensional digital tooth model required for preventing an intersection of the second three-dimensional digital tooth model with the three-dimensional digital antagonistic model due to the second transformation,
determine a largest one of the two shape-deforming adjustments comprising a largest one of the two measures,
adjust the first and second three-dimensional digital tooth model, the adjusting comprising:
   applying in combination the first transformation and the largest shape-deforming adjustment to the first three-dimensional digital tooth model; and
   applying in combination the second transformation and the same largest shape-deforming adjustment to the second three-dimensional digital tooth model.

21. A manufacturing system comprising the computer device of feature combination 20, the manufacturing system further comprising one or more manufacturing devices configured to manufacture the dental restoration,
execution of the program instructions by the processing unit further causing the computer device to:
generate a three-dimensional digital restoration model of the dental restoration comprising the tooth set with the adjusted first and second three-dimensional digital tooth models;
provide data for controlling a manufacturing of a physical dental restoration, the data defining the three-dimensional digital restoration model as a template for the physical dental restoration;
control the one or more manufacturing devices to manufacture the physical dental restauration using the data provided for controlling the manufacturing with the manufactured physical dental restauration being a physical copy of the template defined by the data provided.

### REFERENCE SIGNS LIST

10 computer device
11 manufacturing system
14 external device
16 processing unit
18 bus
20 network adapter
22 I/O interface
24 display
28 memory
RAM
32 cache
34 storage system
40 program
42 program module
50 user interface
52 control elements
54 hardware device
56 keyboard
58 mouse
59 scanner
60 3D printing device
62 printing element
70 machining device
72 machining tool
74 holding device
76 blank
78 raw material
100 3D digital dentition model
106 antagonistic structure
110 first 3D digital tooth model
111 intersection with preparation
112 first surface section
113 first surface point
115 shape-deforming adjustment
114 deformed first surface section
116 first measure
117 first end position of surface
120 first reference direction
122 first line
124 oral direction
126 local coordinate frame
127 coordinate axis
128 coordinate axis
129 coordinate axis
140 second 3D digital tooth model
142 second surface section
143 second surface point
144 deformed second surface section
145 shape-deforming adjustment
146 second measure
147 second end position of surface
150 second reference direction
152 second line
154 oral direction
156 local coordinate frame
157 coordinate axis
158 coordinate axis
159 coordinate axis
160 mirror plane
172 antagonistic jaw
180 intraoral tissue
182 3D digital tissue model
184 3D digital dental restoration model
185 3D digital restoration element
186 dental restoration
188 dental restoration element
[symbol] ₁ first position vector
[symbol] ₂ second position vector
d [symbol] ₁ first position vector after deformation
d [symbol] ₂ second position vector after deformation
Δ₁ first measure
Δ₂ second measure
[symbol] ₁ first reference direction
[symbol] ₂ second reference direction

1. A computer-implemented method for adjusting two or more three-dimensional digital tooth models of a tooth set of three-dimensional digital tooth models of a jaw, the tooth set being a tooth set for a dental restoration, the method comprising:
receiving a three-dimensional digital dentition model comprising the tooth set and defining an arrangement of the three-dimensional digital tooth models of the tooth set, the three-dimensional digital dentition model further comprising a three-dimensional digital antagonistic model of one or more antagonistic structures of an antagonistic jaw and defining an arrangement of the three-dimensional digital antagonistic model relative to the arrangement of the three-dimensional digital tooth models of the tooth set,
pairing a first three-dimensional digital tooth model of the tooth set descriptive of a first tooth with a second three-dimensional digital tooth model of the tooth set descriptive of a second tooth being a contralateral counterpart tooth of the first tooth,
receiving an input defining a first transformation to be applied to the first three-dimensional digital tooth model,
determining for the first transformation a first measure of an additional first shape-deforming adjustment of a shape of the first three-dimensional digital tooth model required for preventing an intersection of the first three-dimensional digital tooth model with the three-dimensional digital antagonistic model due to the first transformation,
determining, using the input, a second transformation to be applied to the second three-dimensional digital tooth model, the determining of the second transformation comprising a mapping of the first transformation to the second three-dimensional digital tooth model,
determining for the second transformation a second measure of an additional second shape-deforming adjustment of a shape of the second three-dimensional digital tooth model required for preventing an intersection of the second three-dimensional digital tooth model with the three-dimensional digital antagonistic model due to the second transformation,
determining a largest one of the two shape-deforming adjustments comprising a largest one of the two measures,
adjusting the first and second three-dimensional digital tooth model, the adjusting comprising:
applying in combination the first transformation and the largest shape-deforming adjustment to the first three-dimensional digital tooth model; and
applying in combination the second transformation and the same largest shape-deforming adjustment to the second three-dimensional digital tooth model.

2. The method of claim 1, the applying of the largest shape-deforming adjustment comprising a mapping of the largest shape-deforming adjustment from an origin three-dimensional digital tooth model to a target three-dimensional digital tooth model, the origin three-dimensional digital tooth model being the one of the first or second three-dimensional digital tooth model, for which the largest shape-deforming adjustment is determined, and the target three-dimensional digital tooth model being the other one of the first or second three-dimensional digital tooth model.

3. The method of claim 2, the mapping of the largest shape-deforming adjustment comprising a mirroring of the largest shape-deforming adjustment onto the target three-dimensional digital tooth model at an adjustment mirror plane arranged between the origin and the target three-dimensional digital tooth model.

4. The method of claim 2, the largest shape-deforming adjustment being mapped to the target three-dimensional digital tooth model, such that a direction of the mapped largest shape-deforming adjustment relative to a target local coordinate frame of the target three-dimensional digital tooth model equals a direction of the largest shape-deforming adjustment relative to an origin local coordinate frame of the origin three-dimensional digital tooth model.

5. The method of claim 1, the first measure being a first distance of a displacing of a first surface section of the first three-dimensional digital tooth model along a first reference direction, which results from the first shape-deforming adjustment, the second measure being a second distance of a displacing of a second surface section of the second three-dimensional digital tooth model along a second reference direction, which results from the second shape-deforming adjustment.

6. The method of claim 5, the first reference direction and the second reference direction being related by a mirror symmetry defined by the adjustment mirror plane.

7. The method of claim 5, a definition of the second reference direction relative to a second local coordinate frame of the second three-dimensional digital tooth model equaling a definition of the first reference direction relative to a first local coordinate frame of the first three-dimensional digital tooth model.

8. The method of claim 5, the first reference direction being a vestibular direction of the first three-dimensional digital tooth model and the second reference direction being a vestibular direction of the second three-dimensional digital tooth model.

9. The method of claim 5, the displacing of the first surface section of the first three-dimensional digital tooth model comprising a displacing of a first vertex of a first mesh defining the first surface section, the displacing of the second surface section of the second three-dimensional digital tooth model comprising a displacing of a second vertex of a second mesh defining the second surface section.

10. The method of claim 9, the first measure being the first distance of the displacing of the first vertex, the second measure being the second distance of the displacing of the second vertex.

11. The method of claim 9, the first and second vertex being corresponding vertices, the method further comprising determining the corresponding first and second vertices, the determining of the corresponding first and second vertices comprising using one of the following: a ray intersection, a closest point determination, an interpolation, a three-dimensional coordinate transformation.

12. The method of claim 9, the displacing of the first surface section comprising a displacing of a first plurality of first vertices of the first mesh comprising the first vertex, the displacing of the second surface section comprising a displacing of a second plurality of second vertices of the second mesh comprising the second vertex,
the determining of the first measure being executed vertex-wise per displaced first vertex of the first plurality of first vertices, the determining of the second measure being executed vertex-wise per displaced second vertex of the second plurality of second vertices,
the determining of the largest shape-deforming adjustment being executed vertex-wise and comprising a comparison of the vertex-wise determined first measure with the corresponding vertex-wise determined second measure,
the applying of the largest shape-deforming adjustment being executed vertex-wise.

13. The method of claim 1, the first transformation comprising one or more of the following: a translation, a rotation, a scaling, a deforming, an adding of tooth material, a removing of tooth material, a modification of a surface structure.

14. The method of claim 1, the mapping of the first transformation comprising a mirroring of the first transformation to the second three-dimensional digital tooth model at a transformation mirror plane arranged between the first and second three-dimensional digital tooth model.

15. The method of claim 1, the three-dimensional digital antagonistic model comprising an antagonistic tooth set of three-dimensional digital antagonistic tooth models of the antagonistic jaw arranged in an antagonistic arrangement.

16. The method of claim 1, the method further comprising:
generating a three-dimensional digital restoration model of the dental restoration comprising the tooth set with the adjusted first and second three-dimensional digital tooth models,
providing data for controlling a manufacturing of a physical dental restoration, the data defining the three-dimensional digital restoration model as a template for the physical dental restoration.

17. The method of claim 16, the method further comprising a manufacturing of the physical dental restoration using the data provided for controlling the manufacturing with the manufactured physical dental restoration being a physical copy of the template defined by the provided data.

18. A computer program product for adjusting two or more three-dimensional digital tooth models of a tooth set of three-dimensional digital tooth models of a jaw, the tooth set being a tooth set for a dental restoration,
the computer program product comprising a non-transitory computer readable storage medium having program instructions embodied therewith, the program instructions being executable by a processing unit of a computer device to cause the computer device to:
receive a three-dimensional digital dentition model comprising the tooth set and defining an arrangement of the three-dimensional digital tooth models of the tooth set, the three-dimensional digital dentition model further comprising a three-dimensional digital antagonistic model of one or more antagonistic structures of an antagonistic jaw and defining an arrangement of the three-dimensional digital antagonistic model relative to the arrangement of the three-dimensional digital tooth models of the tooth set,
pair a first three-dimensional digital tooth model of the tooth set descriptive of a first tooth with a second three-dimensional digital tooth model of the tooth set descriptive of a second tooth being a contralateral counterpart tooth of the first tooth,
receive an input defining a first transformation to be applied to the first three-dimensional digital tooth model,
determine for the first transformation a first measure of an additional first shape-deforming adjustment of a shape of the first three-dimensional digital tooth model required for preventing an intersection of the first three-dimensional digital tooth model with the three-dimensional digital antagonistic model due to the first transformation,
determine, using the input, a second transformation to be applied to the second three-dimensional digital tooth model, the determining of the second transformation comprising a mapping of the first transformation to the second three-dimensional digital tooth model,
determine for the second transformation a second measure of an additional second shape-deforming adjustment of a shape of the second three-dimensional digital tooth model required for preventing an intersection of the second three-dimensional digital tooth model with the three-dimensional digital antagonistic model due to the second transformation,
determine a largest one of the two shape-deforming adjustments comprising a largest one of the two measures,
adjust the first and second three-dimensional digital tooth model, the adjusting comprising:
applying in combination the first transformation and the largest shape-deforming adjustment to the first three-dimensional digital tooth model; and
applying in combination the second transformation and the same largest shape-deforming adjustment to the second three-dimensional digital tooth model.

19. A computer device for adjusting two or more three-dimensional digital tooth models of a tooth set of three-dimensional digital tooth models of a jaw, the tooth set being a tooth set for a dental restoration,
the computer device comprising a processing unit and a memory storing program instructions executable by the processing unit, execution of the program instructions by the processing unit causing the computer device to:
receive a three-dimensional digital dentition model comprising the tooth set and defining an arrangement of the three-dimensional digital tooth models of the tooth set, the three-dimensional digital dentition model further comprising a three-dimensional digital antagonistic model of one or more antagonistic structures of an antagonistic jaw and defining an arrangement of the three-dimensional digital antagonistic model relative to the arrangement of the three-dimensional digital tooth models of the tooth set,
pair a first three-dimensional digital tooth model of the tooth set descriptive of a first tooth with a second three-dimensional digital tooth model of the tooth set descriptive of a second tooth being a contralateral counterpart tooth of the first tooth,
receive an input defining a first transformation to be applied to the first three-dimensional digital tooth model,
determine for the first transformation a first measure of an additional first shape-deforming adjustment of a shape of the first three-dimensional digital tooth model required for preventing an intersection of the first three-dimensional digital tooth model with the three-dimensional digital antagonistic model due to the first transformation,
determine, using the input, a second transformation to be applied to the second three-dimensional digital tooth model, the determining of the second transformation comprising a mapping of the first transformation to the second three-dimensional digital tooth model,
determine for the second transformation a second measure of an additional second shape-deforming adjustment of a shape of the second three-dimensional digital tooth model required for preventing an intersection of the second three-dimensional digital tooth model with the three-dimensional digital antagonistic model due to the second transformation,
determine a largest one of the two shape-deforming adjustments comprising a largest one of the two measures,
adjust the first and second three-dimensional digital tooth model, the adjusting comprising:
applying in combination the first transformation and the largest shape-deforming adjustment to the first three-dimensional digital tooth model; and
applying in combination the second transformation and the same largest shape-deforming adjustment to the second three-dimensional digital tooth model.

20. A manufacturing system comprising the computer device of claim 19, the manufacturing system further comprising one or more manufacturing devices configured to manufacture the dental restoration,
execution of the program instructions by the processing unit further causing the computer device to:
generate a three-dimensional digital restoration model of the dental restoration comprising the tooth set with the adjusted first and second three-dimensional digital tooth models;
provide data for controlling a manufacturing of a physical dental restoration, the data defining the three-dimensional digital restoration model as a template for the physical dental restoration;
control the one or more manufacturing devices to manufacture the physical dental restauration using the data provided for controlling the manufacturing with the manufactured physical dental restauration being a physical copy of the template defined by the data provided.
